(19) United States
(12) Patent Application Publication
(10) Pub. No.: US 2023/0119068 A1
(43) Pub. Date: Apr. 20, 2023

(54) METHODS FOR RAPID SYNTHESIS OF PYRANOANTHOCYANINS

(71) Applicant: Ohio State Innovation Foundation, Columbus, OH (US)

(72) Inventors: Xiaoyi ZHU, Columbus, OH (US); Nicole STRAATHOF, Columbus, OH (US); Gonzalo MIYAGUSUKU-CRUZADO, Columbus, OH (US); M. Monica GIUSTI, Columbus, OH (US); Israel Garcia CANO, Columbus, OH (US); Rafael JIMENEZ-FLORES, Columbus, OH (US)

(21) Appl. No.: 17/799,393

(22) PCT Filed: Feb. 16, 2021
(86) PCT No.: PCT/US2021/018158

§ 371 Date: Aug. 12, 2022

Related U.S. Application Data
(60) Provisional application No. 63/064,115, filed on Aug. 11, 2020.
(60) Provisional application No. 62/976,109, filed on Feb. 13, 2020.

(51) Int. Cl. C07H 17/04; C07D 493/06

(52) U.S. Cl. CPC C07H 17/04; C07D 493/06

(57) ABSTRACT

The present disclosure provides methods for the rapid synthesis of pyranoanthocyanins.

### CROSS-REFERENCE TO RELATED APPLICATIONS

This application claims the benefit of priority to U.S. Provisional Application No. 62/976,109, filed Feb. 13, 2020, and U.S. Provisional Application No. 63/064,115, filed Aug. 11, 2020, the disclosures of each which are incorporated herein by reference in their entirety.

### TECHNICAL FIELD

This disclosure relates to methods for the synthesis of pyranoanthocyanins, and more particularly to methods for the synthesis of pyranoanthocyanins in shortened time periods.

### BACKGROUND

Individuals use color to evaluate food products everyday (Clydesdale, 1993). The color of a product provides information regarding the food's flavor, safety, texture and nutritional value (Sigurdson, Tang, and Giusti, 2017). For example, individuals tend to avoid brown fruits and vegetables in the store because brown is indicative of spoilage and decay. Food companies typically use dyes to color food or enhance the color of products because color is such an important attribute to food. Synthetic colorants are often used because they are stable under a wide range of conditions, are low in cost, and have high tinctorial strength (Cevallos-Casals and Cisneros-Zevallos, 2004). However, there has been increased demand for naturally derived colorants due to consumers having health concerns over synthetic dyes.

Increasing demand for naturally derived colorants has led food manufacturers to explore pigments derived from natural sources. Anthocyanins are pigments with vibrant red, blue and purple hues derived from fruit and vegetable sources (Andersen & Jordhein, 2014). However, anthocyanin pigments are subject to degradation in conditions of varying pH, temperature, or light, as well as in conditions containing large quantities of water or when subjected to bleaching reactions (Cevallos-Casals & Cisneros-Zevallos, 2004). To help stabilize anthocyanins, co-pigmentation and metal complexation have been studied. Further, pigments derived from anthocyanins have been gaining attention,

Pyranoanthocyanins were first found in aged red wine when anthocyanins reacted with yeast metabolites such as pyruvic acid (Somers, 1971; Fulcrand, Cameira dos Santos, Sarni-Manchado, Cheynier, & Favre-Bonvin, 1996; Bakker & Timberlake, 1997). Pyranoanthocyanins are characterized by having an additional ring between C-4 and the C-5 hydroxyl group of the anthocyanin molecule (He et al., 2012), a feature attributed to the enhanced stability of pyranoanthocyanins compared to their anthocyanin counterparts. Pyranoanthocyanins are more resilient to pH changes, water addition and bleaching reactions (Oliveira, Mateus, Silva, and De Freitas, 2009; Oliveira et al., 2006). However, pyranoanthocyanins take significant amounts of time to form (often a month or more), and. the low quantities found naturally limit their application as potential food colorants (Benito, Morato, Palomero, Gonzalez, and Suarez-Lepe, 2011; Morato, Calderon, Gonzalez, Gomez-Cordoves, and Suarez, 2007).

Thus, there is a clear need for methods for the synthesis of pyranoanthocyanins, particularly methods that allow for their preparation in large quantities over shorter time periods.

### SUMMARY

The present disclosure provides methods for the synthesis of pyranoanthocyanins over a period of hours compared to the days or even months required for their synthesis naturally. The disclosed methods allow for the production of larger quantities of pyranoanthocyanins in much shorter time periods, improving their potential applicability as naturally derived pigments.

Thus in one aspect, a method for preparing one or more pyranoanthocyanins of Formula I is provided:

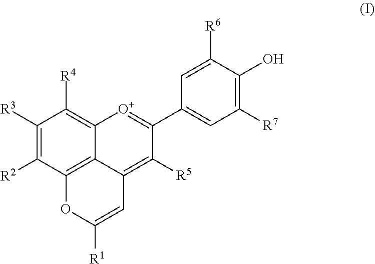

the method comprising:

reacting one or more compounds of Formula II

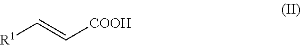

with a bacterium expressing a phenolic acid decarboxylase to form one or more compounds of Formula III

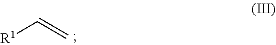

and

reacting the one or more compounds of Formula III with one or more compounds of Formula IV

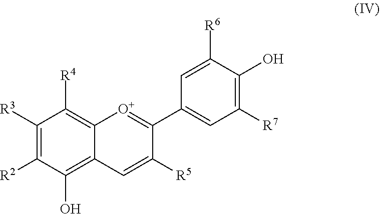

at a temperature ranging from about 35 degrees Celsius to about 45 degrees Celsius to form the one or more pyranoanthocyanins of Formula I;

wherein all variables are as defined herein.

In an alternative aspect, a method for preparing one or more pyranoanthocyanins of Formula I is provided:

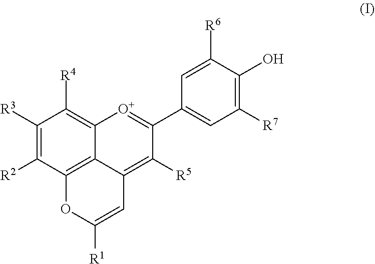

the method comprising:

reacting one or more compounds of Formula II

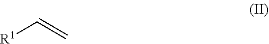

with one or more compounds of Formula IV

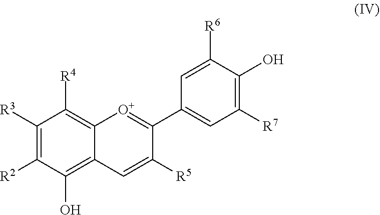

at a temperature ranging from about 30 degrees Celsius to about 80 degrees Celsius to form the one or more pyranoanthocyanins of Formula I;

wherein all variables are as defined herein.

In some embodiments, the compound of Formula IV is reacted with the compound. of Formula II at a molar ratio of about 1:50 to about 1:200 (Formula IV:Formula II). In some preferred embodiments, the molar ratio is about 1:150 (Formula IV:Formula II).

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is a line graph that depicts pyranoanthocyanin formation yields at pH values between 1.0 and 5.0 for day 3, 7, 14, 21 and 30 as described in Example 2. All samples were incubated at 25° C. in the dark. Points are the averages (n=3) and the error bars are the associated standard deviations.

FIG. 2 is a line graph that depicts the percentage of total pigment remaining on days 3, 14 and 30 in relation to day 0 (Eq 3.2) at 12 different pH values as described in Example 2. All samples were incubated at 25° C. in the dark. The averages (n=3) are represented the associated standard deviations are error bars.

FIGS. 3A-3B are bar graphs that depict (3A) The lambda maximum at each pH value and 2 control samples and (3B) the associated absorbance at the lambda maximum for day 0 and day 30 as described in Example 2. Samples were incubated at 25° C. in the dark. Averages are presented (n=3) and the associated standard deviations are represented as error bars.

FIGS. 4A-4C depict: (4A) Chromatogram on day 0 at pH 3.0 and 25° C.; (4B) chromatogram on day 28 at pH 3.0 and 25° C.; and (4C) spectra from 280 nm to 800 nm obtained from the uHPLC as described in Example 2. The solid line is cyanidin-3-glucoside (C3G) spectra and the dashed line is the spectra of the pyranoanthocyanins (PACNs) derived from cyanidin-3-glucoside (C3G) and cyanidin-3-rutinoside (C3R).

FIG. 5 is a table that provides the color properties, based on CIE−L*a*h* and CIE−L*C*_{ab}h_{ab} color systems, on day 0 and day 30 for pH samples between pH 1.0 and 5.0 as described in Example 2. The samples were stored at 25° C. in the dark. Represented as the average (n=3) and the associated standard deviation in parenthesis.

FIGS. 6A-6B are line graphs that depict (6A) percentage of pyranoanthocyanin formation yield and (6B) percentage of the total pigment remaining in relation to day 0 at 5° C., 15° C. and 25° C. in pH 3.0 buffer as described in Example 2. Information was obtained from chromatograms at 500-520 nm. All points are averages (n=3) and error bars are the associated standard deviations.

FIGS. 7A-7B are bar graphs that depict (7A) the lambda maximum for each temperature and (7B) the associated absorbance at the lambda maximum for day 0 and day 28 as described in Example 2. Samples were incubated in the dark at pH 3.0. Averages are presented (n=3) and the associated standard deviations are represented as error bars.

FIG. 8 is a table that provides the color properties, based on CIE−L*a*b* and CIE−L*C*_{ab}h_{ab} color systems, on day 0 and day 28 for 5° C., 15° C. and 25° C. as described in Example 2. Samples were stored in the dark at pH 3.0. Data are represented as the average (n=3) and the associated standard deviation in parenthesis.

FIG. 9 is a bar graph showing pyranoanthocyanins (PACN) formation as a percentage of total pigment from reaction of anthocyanin (ACN) with caffeic acid (CA) as described in Example 5 at 25° C., 50° C., or 75° C. for an incubation period of 3, 6, 24, 48, and 72 hours.

FIG. 10 is a bar graph showing the total pigment remaining under the reaction conditions described in Example 5 at 25° C., 50° C., or 75° C. for an incubation period of 3, 6, 24, 48, and 72 hours.

FIG. 11 is a bar graph showing PACN formation as a percentage of total pigment under the reaction conditions described in Example 5 with ACN:CA molar ratios of 1:0, 1:50, 1:100, 1:150, or 1:200 for an incubation period of 0, 2, 5, 24, 48, 120, or 192 hours.

FIG. 12 is a bar graph showing the absorbance of PACN as a percentage of total pigment remaining under the reaction conditions described in Example 5 with ACN:CA molar ratios of 1:0, 1:50, 1:100, 1:150, or 1:200 for an incubation period of 0, 2, 5, 24, 48, 120, or 192 hours.

FIG. 13 are chromatograms at 500-520 nm showing the absorbances for ACN and PACN after 24 hours incubation when reacted at 25° C., 50° C., and 75° C. with CA and at 75° C. without CA as described in Example 5.

FIGS. 14A and 14B show measurements of UV-Vis light absorption at pH 7 as described in Example 6. FIG. 14A shows absorption spectra of Man, Rogosa and Sharpe broth (MRS), p-coumaric acid (pCA) and 4-vinylphenol (4VP) in chemically defined medium (CDM) or CDM alone. In FIG. 14B, absorption at 260, 300 and 340 nm showed that MRS had a significantly higher absorption than CDM did, between 200-500 n. Different letters indicate significant differences (p<0.01). Data represents means of n=3, error bars represent SD.

FIG. 15 shows UV-Vis absorption spectra of pCA: p-coumaric acid, CA: caffeic acid, FA: ferulic acid, 4VP: 4-vinylphenol, 4VC: 4-vinylcatechol, MRS: De Man, Rogosa and Sharpe broth, CDM: chemically defined medium, diluted 25 times in buffers at different pH values. Bathochromic shifts were observed in the λ_{230-500 max} of HCAs when pH decreased. No changes in the _230-500 max of 4VPs were observed. Light absorption of MRS was higher than CDM at all pH values. Data represent means of n=3, error bars represent SD.

FIG. 16 shows ultra-high-pressure liquid chromatography-mass spectrometry representative chromatograms and identification of the initial hydroxycinnamic acids and their respective decarboxylated products after incubation for 36 h at 32° C. in the dark with Lactobacillus plantarum (OSU-PECh-BB). λₘₐₓ: maximum absorption in the 230-500 nm range.

FIG. 17 shows representative UV-Vis light absorption spectra for hydroxycinnamic acids and decarboxylated products using a diode-array detector (upper) and a plate reader (lower) after incubation with Lactobacillus plantarum (OSU-PECh-BB). Spectra for pCA and 4VP (blue), CA and 4VC (purple), and FA and 4VG (orange). Spectra read on CDM diluted with pH 1.0 buffer using a plate reader.

FIG. 18 shows UV-Vis light absorption changes for p-coumaiic acid (upper) and caffeic acid (lower) during incubation with Enterococcus mundtii (OSU-PECK-39B), Pediococcus pentosaceus (OSU-PECh-27B). Lactobacillus plantarum (OSU-PECh-BB) and Lactobacillus casei (OSU-PECh-C) for up to six hours at 32° C. in the dark. Data represent means of n=3, bars represent SD.

Like reference symbols in the various drawings indicate like elements.

### DETAILED DESCRIPTION

Many modifications and other embodiments disclosed herein will come to mind to one skilled in the art to which the disclosed compounds, compositions and methods pertain having the benefit of the teaching presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of the disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from and combined with the features of any of the other several embodiments without departing from the scope and spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuations, or the number or type of aspects described in the specification.

All publications mentioned herein are incorporated herein by reference to disclose and described the methods and/or materials in connection with which the publications are cited. The publications discussed herein are provided solely for the disclosure prior to the filing date of the present application. The dates of publication provided herein can be different from the actual publication dates, which can require independent confirmation.

It is also to be understood that the terminology used herein is for the purpose of describing particular aspects only and is not intended to be limited. Unless defined otherwise, all technical and scientific terms used herein have the same meaning as commonly understood by one of ordinary skill in the art to which the disclosed compounds, compositions, and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated feature, integers, steps, or components as referred to, but does not preclude the presence or addition of one or more features, integers, steps, or components, or groups thereof. Moreover, each of the terms “by”, “comprising”, “comprises”, “comprised of”, “including”, “includes”, “included”, “involving”, “involves”, “involved”, and “such as” are used in their open, non-limiting sense and may be used interchangeably. Further, the term “comprising” is intended to include examples and aspects encompassed by the terms “consisting essentially of” and “consisting of”. Similarly, the term “consisting essentially of” is intended to include examples encompassed by the term “consisting of”.

As used in the specification and the appended claims, the singular forms “a”, “an” and “the” include plural referents unless the context clearly dictates otherwise. Thus, for example, reference to “a compound”, “a bacterium”, or “an anthocyanin” includes, but is not limited to, two or more such compounds, bacteria, or anthocyanins, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further aspect. For example, if the value “about 10” is disclosed, then “10” is also disclosed.

As used herein, the terms “about”, “approximate”, “at or about”, and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably be determined. In such cases, it is generally understood, as used herein, that “about” and “at or about” mean the nominal value indicated ±10% variation unless otherwise indicated or inferred. In general, an amount, size, formulation, parameter, or other quantity or characteristic is “about”, “approximate”, or “at or about” whether or not expressly stated to be such. it is understood that where “about”, “approximate”, or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context.

A dash (—) that is not between two letters or symbols is used to indicate a point of attachment for a substituent. For example, —(C═O)NH₂ is attached through the carbon of the keto (C═O) group,

The present disclosure provides methods for preparing one or more pyranoanthocyanins of Formula I

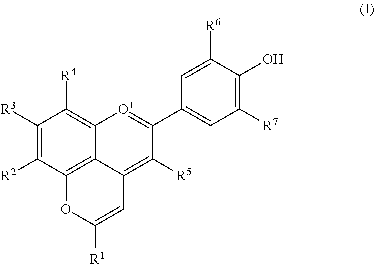

wherein:

R¹ is selected from the group consisting of:

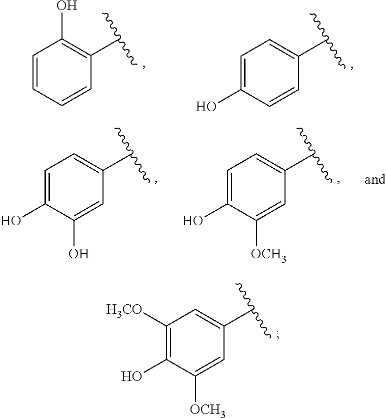 [SMILES: *c1ccccc1O ; *c1ccc(O)cc1 ; *c1ccc(O)c(O)c1 ; *c1ccc(O)c(OC)c1 ; *c1cc(OC)c(O)c(OC)c1]

R² is selected from —H and —OCH₃;

R³ is selected from —OH and —OCH₃;

R⁴ is selected from —H and —OCH₃;

R⁵ is selected from —OH, —O-acyl, and a non-acylated or acylated O-glycoside;

R⁶ is selected from —H, —OH, and —OCH₃; and

R⁷ is selected from —H, —OH, and —OCH₃.

In one aspect, the method comprises:

reacting one or more compounds of Formula II

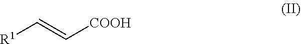

with a bacterium expressing a phenolic acid decarboxylase to form one or more compounds of Formula III

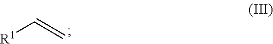

and

reacting the one or more compounds of Formula II with one or more compounds of Formula IV

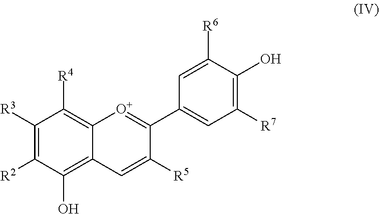

at a temperature ranging from about 35 degrees Celsius to about 45 degrees Celsius to form the one or more pyranoanthocyanins of Formula I.

In an alternative aspect, the method comprises:

reacting one or more compounds of Formula II

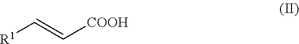

with one or more compounds of Formula IV

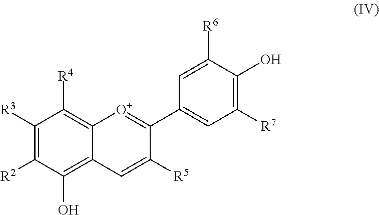

at a temperature ranging from about 30 degrees Celsius to about 80 degrees Celsius to form the one or more pyranoanthocyanins of Formula I.

In some embodiments, the compound of Formula II comprises

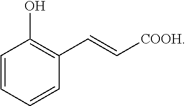 [SMILES: O=C(O)/C=C/c1ccccc1O]

In some embodiments, the compound of Formula II comprises

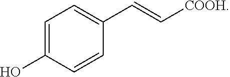 [SMILES: O=C(O)/C=C/c1ccc(O)cc1]

In some embodiments, the compound of Formula II comprises

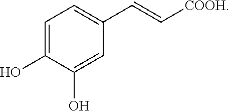 [SMILES: O=C(O)/C=C/c1ccc(O)c(O)c1]

In some embodiments, the compound of Formula II comprises

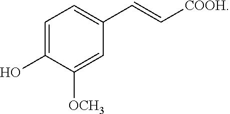 [SMILES: COc1cc(/C=C/C(=O)O)ccc1O]

In sonic embodiments, the compound of Formula II comprises

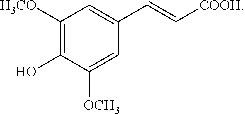 [SMILES: COc1cc(/C=C/C(=O)O)cc(OC)c1O]

In some embodiments, the compound of Formula III comprises

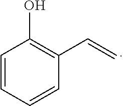

In some embodiments, the compound of Formula III comprises

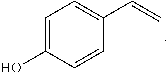

In some embodiments, the compound of Formula III comprises

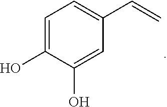

In some embodiments, the compound of Formula III comprises

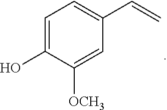

In some embodiments, the compound of Formula III comprises

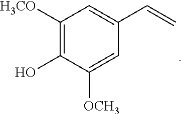

In some embodiments of Formula I or Formula IV, R² is —H. In some embodiments of Formula I or Formula IV, R² is —OCH₃.

In some embodiments of Formula I or Formula IV, R³ is —OH. In some embodiments of Formula I or Formula IV, R³ is —OCH₃.

In some embodiments of Formula I or Formula IV, R⁴ is —H. In some embodiments of Formula I or Formula IV, R⁴ is —OCH₃.

In some embodiments of Formula I or Formula IV, R⁶ is —H. In some embodiments of Formula I or Formula IV, R⁶ is —OH. In some embodiments of Formula I or Formula IV, R⁶ is —OCH₃.

In some embodiments of Formula I or Formula IV, R⁷ is —H. In some embodiments of Formula I or Formula IV, R⁷ is —OH. In some embodiments of Formula I or Formula IV, R⁷ is —OCH₃.

In some embodiments, the compound of Formula IV comprises a compound selected from the group consisting of:

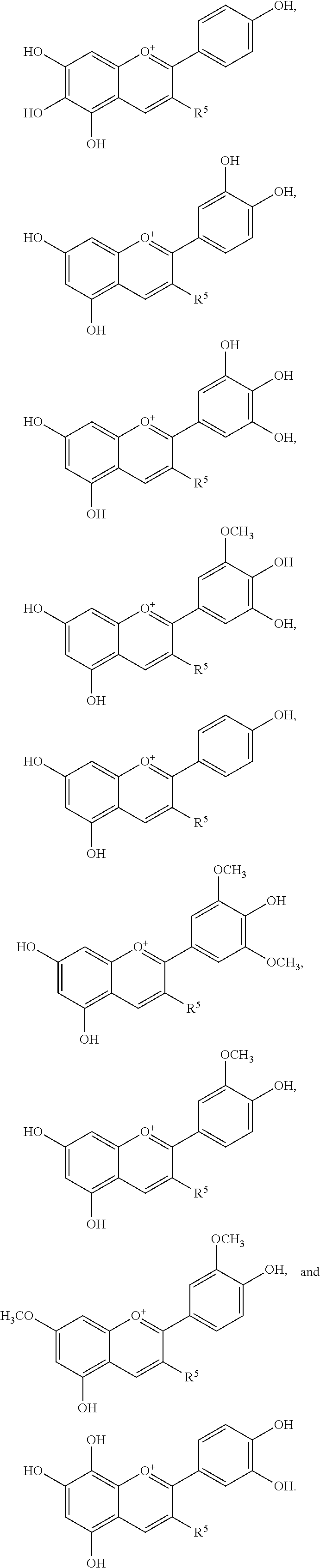

In some embodiments, the pyranoanthocyanin of Formula I comprises a compound selected from the group consisting of:

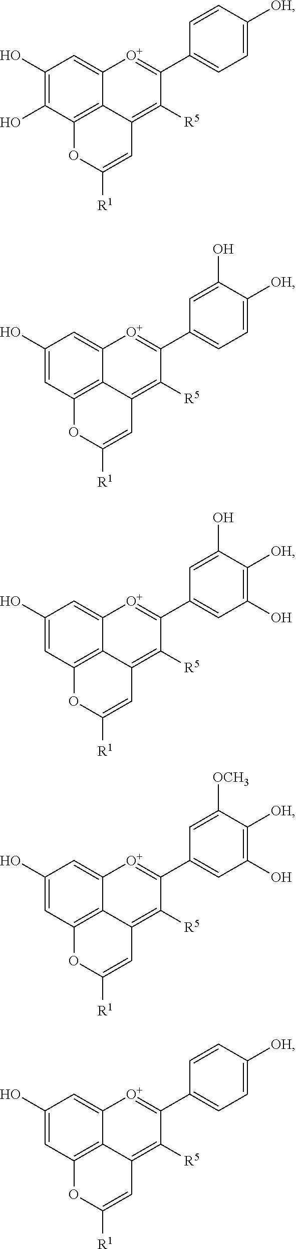

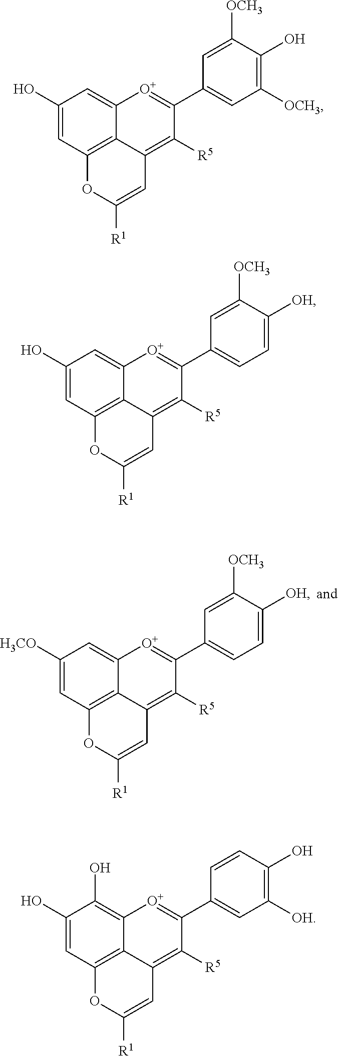

In some embodiments, the pyranoanthocyanin of Formula I comprises a compound selected from the group consisting of:

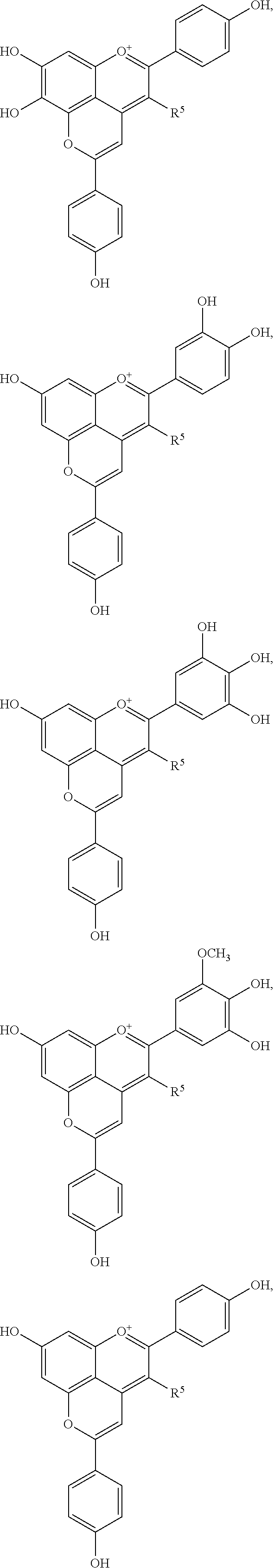

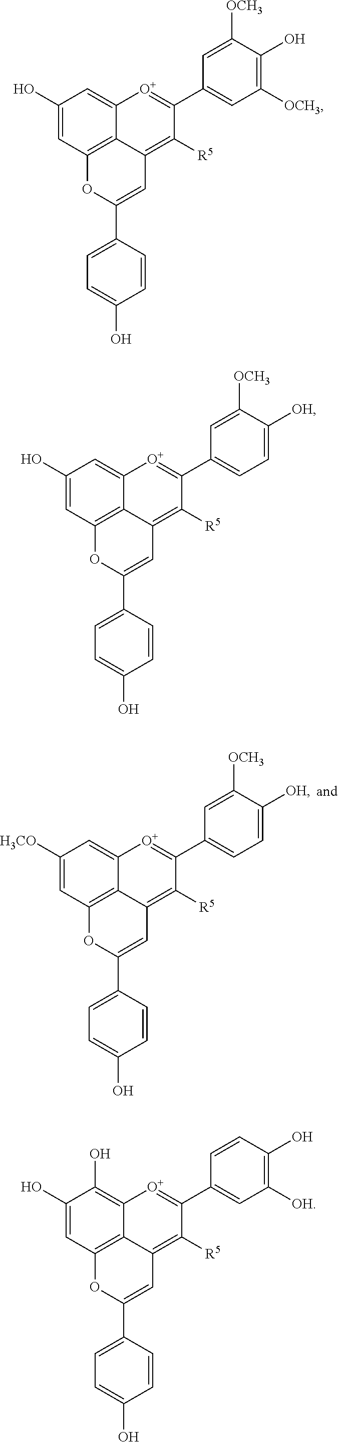

In some embodiments, the pyranoanthocyanin of Formula I comprises a compound selected from the group consisting of:

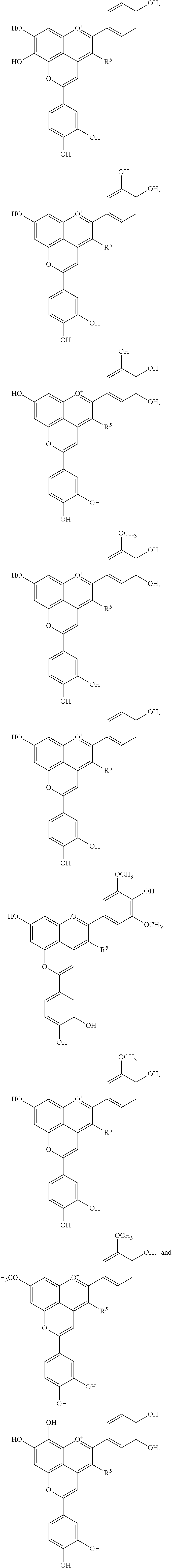

In some embodiments, the pyranoanthocyanin of Formula I comprises a compound selected from the group consisting of:

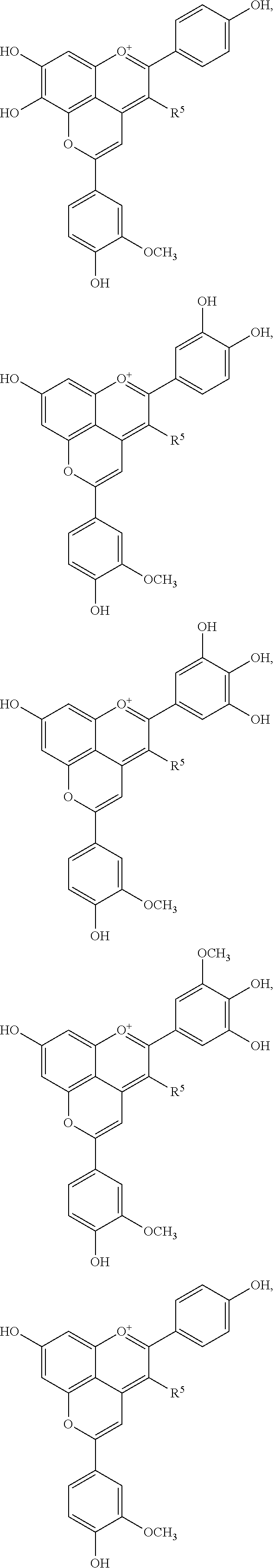

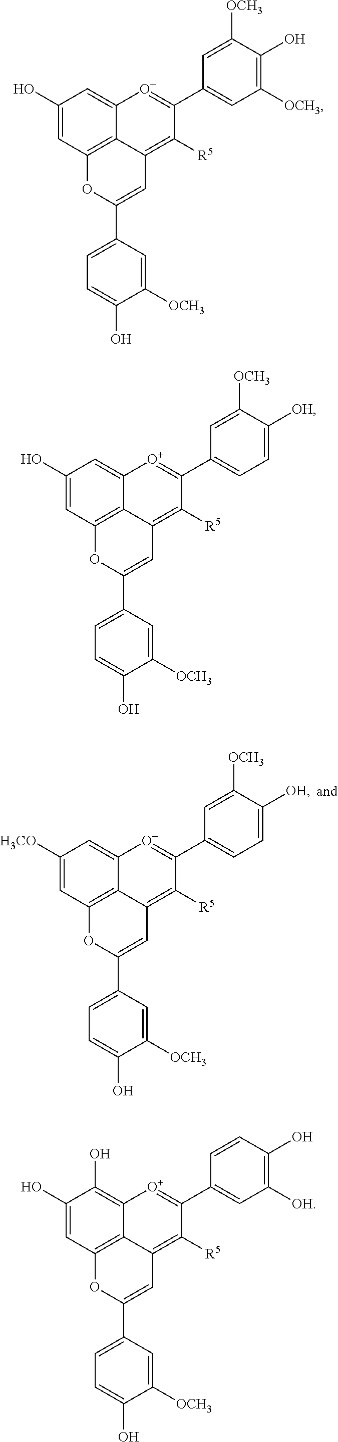

In some embodiments of Formula I or Formula IV, R⁵ is —O-acetyl having the chemical structure:

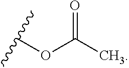 [SMILES: *OC(C)=O]

In some embodiments of Formula I or Formula IV, R⁵ is —O-malonyl having the chemical structure:

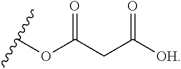 [SMILES: *OC(=O)CC(=O)O]

In some embodiments of Formula I of Formula IV, R⁵ is —O-succinyl having the chemical structure:

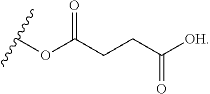 [SMILES: *OC(=O)CCC(=O)O]

In some embodiments of Formula I or Formula IV, R⁵ is —O-(4-hydroxybenzoyl having the chemical structure:

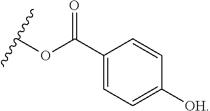 [SMILES: *OC(=O)c1ccc(O)cc1]

In some embodiments of Formula I or Formula IV, R⁵ is —O-(3,4-dihydroxybenzoyl) having the chemical structure:

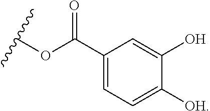 [SMILES: *OC(=O)c1ccc(O)c(O)c1]

In some embodiments of Formula I or Formula IV, R⁵ is —O-(vanilloyl) having the chemical structure:

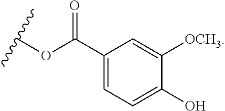 [SMILES: *OC(=O)c1ccc(O)c(OC)c1]

In some embodiments of Formula I or Formula IV, R⁵ is —O-(galloyl) having the chemical structure:

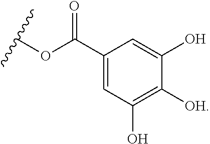 [SMILES: *OC(=O)c1cc(O)c(O)c(O)c1]

In some embodiments of Formula I or Formula IV, R⁵ is —O-(syringoyl) having the chemical structure:

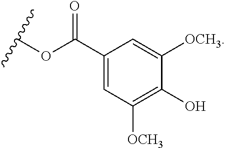 [SMILES: *OC(=O)c1cc(OC)c(O)c(OC)c1]

In some embodiments of Formula I or Formula IV, R⁵ is —O-(2-coumaryl) having the chemical structure:

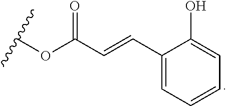 [SMILES: *OC(=O)/C=C/c1ccccc1O]

In some embodiments of Formula I or Formula IV, R⁵ is —O-(4-coumaryl) having the chemical structure:

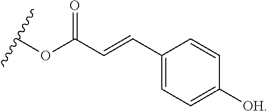 [SMILES: *OC(=O)/C=C/c1ccc(O)cc1]

In some embodiments of Formula I or Formula IV, R⁵ is —O-(caffeoyl) having the chemical structure:

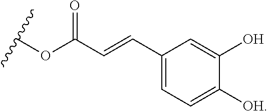 [SMILES: *OC(=O)/C=C/c1ccc(O)c(O)c1]

In some embodiments of Formula I or Formula IV, R⁵ is —O-(feruloyl) having the chemical structure:

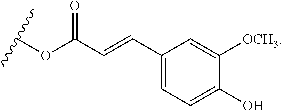 [SMILES: *OC(=O)/C=C/c1ccc(O)c(OC)c1]

In some embodiments of Formula I or Formula IV, R⁵ is —O-(sinapoyl) having the chemical structure:

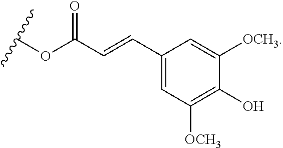 [SMILES: *OC(=O)/C=C/c1cc(OC)c(O)c(OC)c1]

In some embodiments of Formula I or Formula IV, R⁵ is arabinoside having the chemical structure:

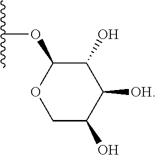 [SMILES: *O[C@@H]1OC[C@H](O)[C@H](O)[C@H]1O]

In some embodiments of Formula I or Formula IV R⁵ is galactoside having the chemical structure:

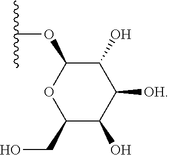 [SMILES: *O[C@@H]1O[C@H](CO)[C@H](O)[C@H](O)[C@H]1O]

In sonic embodiments of Formula I or Formula IV, R⁵ is glucoside having the chemical structure:

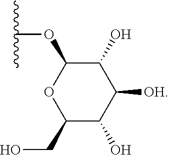 [SMILES: *O[C@@H]1O[C@H](CO)[C@@H](O)[C@H](O)[C@H]1O]

In some embodiments of Formula I or Formula IV, R⁵ is glucosyl(1→2)glucoside having the chemical structure:

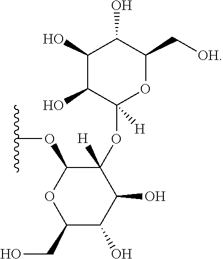 [SMILES: *O[C@@H]1O[C@H](CO)[C@@H](O)[C@H](O)[C@H]1O[C@@H]1O[C@H](CO)[C@@H](O)[C@H](O)[C@@H]1O]

In some embodiments of Formula I or Formula R⁵ is rhamnosyl(1→6)glucoside having the chemical structure:

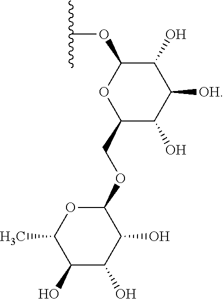 [SMILES: *O[C@@H]1O[C@H](CO[C@@H]2O[C@@H](C)[C@H](O)[C@@H](O)[C@H]2O)[C@@H](O)[C@H](O)[C@H]1O]

In some embodiments of Formula I or Formula IV, R⁵ is xylosyl(1→2)galactoside having the chemical structure:

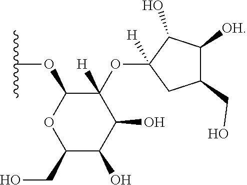 [SMILES: *O[C@@H]1O[C@H](CO)[C@H](O)[C@H](O)[C@H]1O[C@@H]1C[C@H](CO)[C@H](O)[C@H]1O]

In some embodiments of Formula I or Formula IV, R⁵ is xylosyl(1→2)glucoside having the chemical structure:

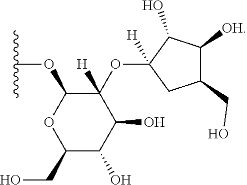 [SMILES: *O[C@@H]1O[C@H](CO)[C@@H](O)[C@H](O)[C@H]1O[C@@H]1C[C@H](CO)[C@H](O)[C@H]1O]

In some embodiments of Formula I or Formula IV, R⁵ is glucosyl(1→6)galactoside having the chemical structure:

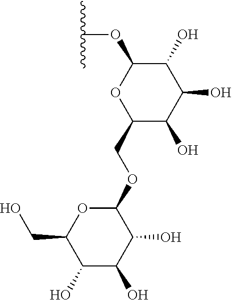 [SMILES: *O[C@@H]1O[C@H](CO[C@@H]2O[C@H](CO)[C@@H](O)[C@H](O)[C@H]2O)[C@H](O)[C@H](O)[C@H]1O]

In some embodiments of Formula I or Formula IV, R⁵ is xylosyl(1→2)glucosyl(1→6)galactoside having the chemical structure:

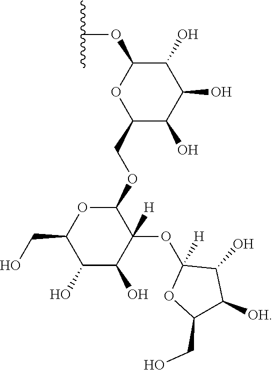 [SMILES: *O[C@@H]1O[C@H](CO[C@@H]2O[C@H](CO)[C@@H](O)[C@H](O)[C@H]2O[C@@H]2O[C@H](CO)[C@H](O)[C@H]2O)[C@H](O)[C@H](O)[C@H]1O]

In some embodiments of Formula I or Formula IV, R⁵ is selected from arabinoside, galactoside, glucoside, glucosyl(1→2)glucoside, rhamnosyl(1→6)glucoside, xylosyl(1→2)galactoside, xylosyl(1→2)glucoside, glucosyl(1→6)galactoside, and xylosyl(1→2)glucosyl(1→6)galactoside substituted on one or more of hydroxyl groups in R⁵ with an acyl group (for example one, two, three or four acyl groups) independently selected at each occurrence from:

acetyl having the chemical structure

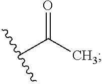 [SMILES: *C(C)=O]

malonyl having the chemical structure

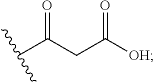 [SMILES: *C(=O)CC(=O)O]

succinyl having the chemical structure

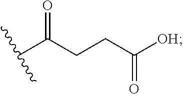 [SMILES: *C(=O)CCC(=O)O]

4-hydroxybenzoyl having the chemical structure

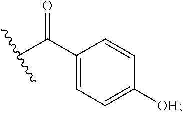 [SMILES: *C(=O)c1ccc(O)cc1]

3,4-dihydroxybenzoyl having the chemical structure

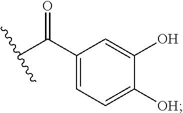 [SMILES: *C(=O)c1ccc(O)c(O)c1]

vanilloyl having the chemical structure

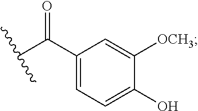 [SMILES: *C(=O)c1ccc(O)c(OC)c1]

galloyl having the chemical structure

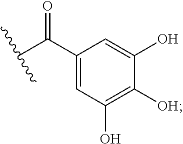 [SMILES: *C(=O)c1cc(O)c(O)c(O)c1]

syringoyl having the chemical structure

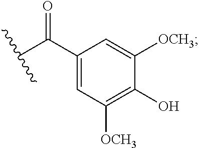 [SMILES: *C(=O)c1cc(OC)c(O)c(OC)c1]

2-coumaryl having the chemical structure

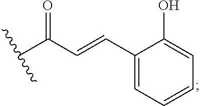 [SMILES: *C(=O)/C=C/c1ccccc1O]

4-coumaryl having the chemical structure

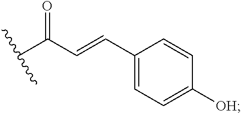 [SMILES: *C(=O)/C=C/c1ccc(O)cc1]

caffeoyl having the chemical structure

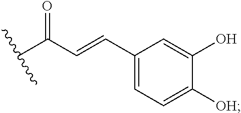 [SMILES: *C(=O)/C=C/c1ccc(O)c(O)c1]

feruloyl having the chemical structure

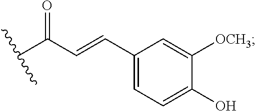 [SMILES: *C(=O)/C=C/c1ccc(O)c(OC)c1]

and

sinapoyl having the chemical structure

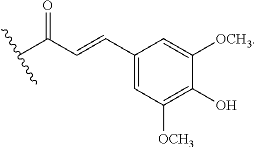 [SMILES: *C(=O)/C=C/c1cc(OC)c(O)c(OC)c1]

In one aspect, the bacterium as used in the present method expresses a phenolic acid decarboxylase (PAD). The bacterium may naturally express the PAD enzyme or may comprise a bacterium containing a recombinant plasmid which encodes for the PAD enzyme. In some embodiments, the bacterium may express a phenolic acid decarboxylase having at least 70% sequence identity with PadC (UniProtKb-O07006).

In some embodiments, the bacterium which expresses a phenolic acid decarboxylase comprises a lactic acid bacterium. Lactic acid bacteria are an order of Gram-positive, low-GC, acid-tolerant, generally nonsporulating, nonrespiring, either rod shaped (bacilli) or spherical (cocci) bacteria that share common metabolic and physiological characteristics. These bacteria, usually found in decomposing plants and milk products, produce lactic acid as the major metabolic end product of carbohydrate fermentation. This trait has, throughout history, linked lactic acid with food fermentations, as acidification inhibits the growth of spoilage agents. Furthermore, lactic acid and other metabolic products contribute to the organoleptic and textural profile of a food item. The industrial importance of the lactic acid bacteria is further recognized by their Generally Recognized as Safe (GRAS) status, due to their ubiquitous appearance in food and their contribution to the healthy microbiota of animal and human mucosal surfaces. Although many genera of bacteria produce lactic acid as primary or secondary fermentation products, typical lactic acid bacterium are those of the Lactobacillales order, including the following genera: Lactobacillus, Carnobacterium, Lactococcus, Streptococcus, Enterococcus, Vagococcus, Leuconostoc, Oenococcus, Pediococcus, Tetragonococcus, Aerococcus, and Weissella.

In some embodiments, the lactic acid bacterium may comprise a Lactobacillus species, for example Lactobacillus acidophihus, Lactobacillus brevis, Lactobacillus casei, Lactobacillus crispatus, Lactobacillus delbruekii ssp. bulgaricus, Lactobacillus fermentum, Lactobacillus gasseri, Lactibacillus helveticus, Lactobacillus jensenii, Lactobacillus johnsonii, Lactobacillus keferi, Lactobacillus mesenteroides, Lactobacillus paracasei, Lactobacillus plantarum, and Lactobacillus reuteri, Lactobacillus salivarius, or Lactobacillus rhamnosus. In some embodiments, the lactic acid bacterium may comprise a Lactococcus species, for example Lactococcus lactic, Lactococcus lactis ssp. cremoris, or Lactococcus lactis ssp. lactis. In some embodiments, the lactic acid bacterium may comprise a Streptococcus species, for example Streptococcus thermophilus. In some embodiments, the lactic acid bacterium may comprise a Leuconostoc species, for example Leuconostoc lactis. In some embodiments, the lactic acid bacterium may comprise an Enterococcus species, for example Enterococcus faecum or Enterococcus mundtii. In some embodiments, the lactic acid bacterium may comprise a Pediococcus species, for example Pediococcus cerevisiae, and Pediococcus pentosaceus.

In some embodiments, the bacterium may comprise a Bifidobacterium species, for example Bifidobacterium adolescentis, Bifidobacterium breve, Bifidobacterium longum, Bifidobacterium animalis, Bifidobacterium infantis, Bifidobacterium thermophilum, Bifidobacterium bifidum, or Bifidobacterium lactis.

In some embodiments, the one or more compounds of Formula III and the one or more compounds of Formula IV are reacted at a temperature of about 35 degrees Celsius, about 36 degrees Celsius, about 37 degrees Celsius, about 38 degrees Celsius, about 39 degrees Celsius, about 40 degrees Celsius, about 41 degrees Celsius, about 42 degrees Celsius, about 43 degrees Celsius, about 44 degrees Celsius, or about 45 degrees Celsius.

In some embodiments, the one or more compounds of Formula III and the one or more compounds of Formula IV are reacted at a pH from about 2 to about 4, for example from about 2.5 to about 3.5 or from about 2.8 to about 3.5. In some embodiments, the one or more compounds of Formula III and the one or more compounds of Formula IV are reacted at a pH of about 2.0, about 2.1, about 2.2, about 2.3, about 2.4, about 2.5, about 2.6, about 2.7, about 2.8, about 2.9, about 3.0, about 3.1, about 3.2, about 3.3. about 3.4, about 3.5, about 3.6, about 3.7, about 3.8, about 3.9 or about 4.0.

In some embodiments, the compound of Formula I may be formed by reaction of the compound of Formula III and the compound of Formula IV as greater than 50% of the content of the total pigment in the reaction mixture in 24 hours, 20 hours, 18 hours, 16 hours, 14 hours, 12 hours, 6 hours, 4 hours or less.

In sonic embodiments, the one or more compounds of Formula II and the one or more compounds of Formula IV are reacted at a temperature of about 30 degrees Celsius, about 32 degrees Celsius, about 34 degrees Celsius, about 36 degrees Celsius, about 38 degrees Celsius, about 40 degrees Celsius, about 42 degrees Celsius, about 44 degrees Celsius, about 46 degrees Celsius, about 48 degrees Celsius, about 50 degrees Celsius, about 52 degrees Celsius, about 54 degrees Celsius, about 56 degrees Celsius, about 58 degrees Celsius, about 60 degrees Celsius, about 62 degrees Celsius, about 64 degrees Celsius, about 66 degrees Celsius, about 68 degrees Celsius, about 70 degrees Celsius, about 72 degrees Celsius, about 74 degrees Celsius, about 76 degrees Celsius, about 78 degrees Celsius, or about 80 degrees Celsius.

In some embodiments, the one or more compounds of Formula II and the one or more compounds of Formula IV are reacted at a pH from about 2 to about 4, for example from about 2.5 to about 3.5 or from about 2.8 to about 3.5. In some embodiments, the one or more compounds of Formula II and the one or more compounds of Formula IV are reacted at a pH of about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9 or about 4.0.

In some embodiments, the compound of Formula IV is reacted with the compound. of Formula II at a molar ratio of about 1:50 to about 1:200 (Formula IV:Formula II) for example about 1:75, about 1:100, about 1:125, about 1:150, or about 1:175. In some preferred embodiments, the molar ratio is about 1:150 (Formula IV:Formula II).

In some embodiments, the compound of Formula I may be formed by reaction of the compound of Formula II and the compound of Formula IV as greater than 50% of the content of the total pigment in the reaction mixture in 72 hours, 48 hours, 36 hours, 24 hours, or less.

In some embodiments wherein the compound of Formula I is acylated with a cinnamyl derivative, the method further comprises irradiating the compound of Formula I with ultraviolet (UV) light. Irradiation with UV light results in conversion of the cinnamyl substituent from a trans configuration to a cis configuration. Such a conversion may be desirable to broaden the range of colors provided or to increase stability.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

### Example 1. Decarboxylation of hydroxycinnamic acids by lactic acid Bacteria Strains Isolated from Dairy Products

The decarboxylating activity of lactic acid bacteria (LAB) strains (137 strains) with potential to produce phenolic acid decarboxylase (22 strains) was evaluated after incubation with hydroxycinnamic acids (HCAs) for 36 hours at 32° C. in the dark. Decarboxylation was monitored using a high-throughout spectrophotometric method based on the observed hypsochromic shifts upon conversion to 4-vinylphenol compounds. Spectrophotometric results were confirmed by HPLC-DAD-MS/MS analyses, looking for longer retention times and shorter λ_{230-500 max} than their precursor HCA, and characteristic m/z. Enterococcus mundtii, Lactobacillus plantarum, and Pediococcus pentosaceus were capable of decarboxylating p-coumaric, caffeic and ferulic acids, producing their 4-vinylphenol derivatives. Another seven strains were only capable of biotransforming p-coumaric and caffeic acid, one was able to decarboxylate only caffeic acid and one was able to dicarboxylic only p-coumaric acid, while ten strains were not able to biotransform any HCA. No strain examined was capable of decarboxylating sinapic acid. p-Coumaric acid had the highest biotransformation efficiency, followed by caffeic acid and lastly ferulic acid. No reductase activity by the LAB strains evaluated was detected, as evidenced by the absence of 4-ethylphenols.

### Example 2. The Effects of pH and Temperature on Formation of pyranoanthocyanins from cyanidin-3-glucoside and cyanidin-3-rutinoside Over Time

### Materials and Methods

Blackberries (Rubus sp., SUNBELLE, Los Reyes, Mexico) were purchased from a local grocery store. Lab grade pyruvic acid was purchased from Sigma Aldrich (St. Louis, Mo., U.S.). Lab grade acetaldehyde, ACS grade acetone and HPLC grade chloroform were purchased from Fisher Scientific (Hampton, N.H., U.S.). HPLC-MS grade acetonitrile and HPLC-MS grade water were obtained from Fisher Scientific (Hampton, N.H., U.S.). HPLC grade formic acid was purchased from Sigma Aldrich (St. Louis, Mo., U.S.)

### Anthocyanin Extraction and Preparation

Fresh blackberries were used as the starting material. Extraction procedures were conducted according to procedures described by Rodriguez-Saona & Wrolstad (2001). Blackberry anthocyanins are predominantly cyanidin-3-glucoside (C3G) and cyanidin-3-rutinoside (C3R) with small amounts of cyanidin-3-malonylglucoside and cyanidin-3-dioxalyglucoside (Fan-Chiang, & Wrolstad, 2005). The blackberry extract was saponified with 10% KOH for 10 minutes and acidified with 2N HCl and purified by a C18-resin (Waters Corporation, Milford, Mass., U.S.) using solid phase extraction (SPE) (Rodriguez-Saona & Worlstad 2001). The sample was washed with two volumes of 0.01% HCl water and three volumes of ethyl acetate. The sample was eluted with methanol. The residual methanol was evaporated with a rotary evaporator (BUCHI Rotavapor Collegiate Evaporation System, BUCHI Corporation, New Castle, Del., U.S.) under vacuum at 35° C. The purified pigment was resolubilized in 0.01% HCl water. The final anthocyanin composition was cyanidin-3-glucoside (93.0%) and cyanidin-3-rutinoside (7.0%), according to results by HPLC monitoring described below.

### Anthocyanin and Pyranoanthocyanin Monitoring

A uHPLC (Shimadzu Nexera-I LC-2040C, Columbia, Md., U.S.) was used to monitor changes in anthocyanin composition and pyranoanthocyanin formation. A flow rate of 0.4 mL/min was used, and the column oven was set to 60° C. The injection volume for all samples was 5.0 μL. A Pinnacle DB (Restek Corporation, Bellefonte, Pa., U.S.) C18 column (1.9 μM particle size, 50×2.1 mm length) was used. Both the control and treatment samples were ran with the following gradient of Solvent A (4.5% formic acid water) and Solvent B (100% Acetonitrile): 3% B from 0.00 seconds to 1 minute, ramped to 4.3% B by 7.00 minutes, ramped to 40% B by 7.50 minutes, held at 40% B until 8 minutes, decreased to 3% B by 8.50 minutes, and held at 3% B until 10 minutes. All samples were analyzed by uHPLC during 1 month of storage in the dark. A standard curve was created using cyanidin-3-glucoside (Sigma Aldrich, St. Louis, Mo., U.S.).

### UV-Vis Spectrophotometry and Colorimetry

The absorbance spectra of all samples were collected over a month storage using a micro-well plate reader (SpectraMax 190, Molecular Devices, Sunnyvale, Calif., U.S.). Sample aliquots of 50 μL were loaded into a 96-well plate. Samples were evaluated from 380 nm to 720 nm in 1 nm intervals. Distilled water was used as a blank.

Colorimetric data was obtained using a software, ColorBySpectra, that converted the spectral data into colorimetric data (Farr & Giusti, 2017). Color data was obtained with regular transmission, 1964 standard equations, D₆₅ illuminant, and 10° observer angle. Color values were reported using CIE-L*a*b* and CIE-L*C*_{ab}h_{ab} color systems.

### Temperature Sample Preparation

The anthocyanin concentration was determined using a standard curve of cyanidin-3-glucoside (Sigma Aldrich, St. Louis, Mo., U.S.) at an AUC of 500-520 nm. The anthocyanins were diluted in buffer solutions to obtain final concentrations of 500 μM. The buffer was a 0.2 M citrate buffer (citric acid and sodium citrate dihydrate) adjusted to pH 3.0 with HCl and NaOH. A 0.2 M buffer was used to better control the effect temperature had on pH. A stock solution of pyruvic acid was made using pyruvic acid and the citrate buffer. Pyruvic acid was added at a 200× molar ratio of anthocyanin to pyruvic acid (100 mM). 2 mL of all samples were made, and the samples were filtered through a 0.2 μM membrane into 2 mL HPLC vials. Control samples with no pyruvic acid and the treatment samples were stored in the dark for 28 days in 5° C., 15° C., and 25° C. incubators. All samples were prepared and ran in triplicate.

### pH Sample Preparation

The anthocyanins were diluted with a 0.1 M phosphate buffer to obtain a concentration of 500 μM for all samples. The phosphate buffers were adjusted to pH 1.0, 2.0, 2.5, 3.0, 3.5, 4.0 and 5.0, prior to dilution. A pyruvic acid stock solution was made using pyruvic acid and HPLC water. Pyruvic acid was added to obtain 200× the anthocyanin concentration on a molar ratio (100 mM). The pH values were evaluated with a S220 SevenCompact pH meter (Mettler Toledo, Columbus, Ohio, U.S.). The pH of the control samples remained within ±0.05 of the initial pH throughout the study. When pyruvic acid was added to the samples, the pH of the solutions shifted, and treatment samples had pH values of 1.0, 1.8, 2.0, 2.3, 2.6. 2.8, 3.1. 3.5, 3,6, 3.8, 4.1 and 5.0. All pH values varied by less than 0.05 throughout the study, All samples were filtered through a 0.2 μM membrane into 2 mL HPLC vials and stored in the dark for 30 days at 25° C. All samples were prepared and ran in triplicate.

### Results and Discussion

### Effect of pH on Pyranoanthocyanin Formation

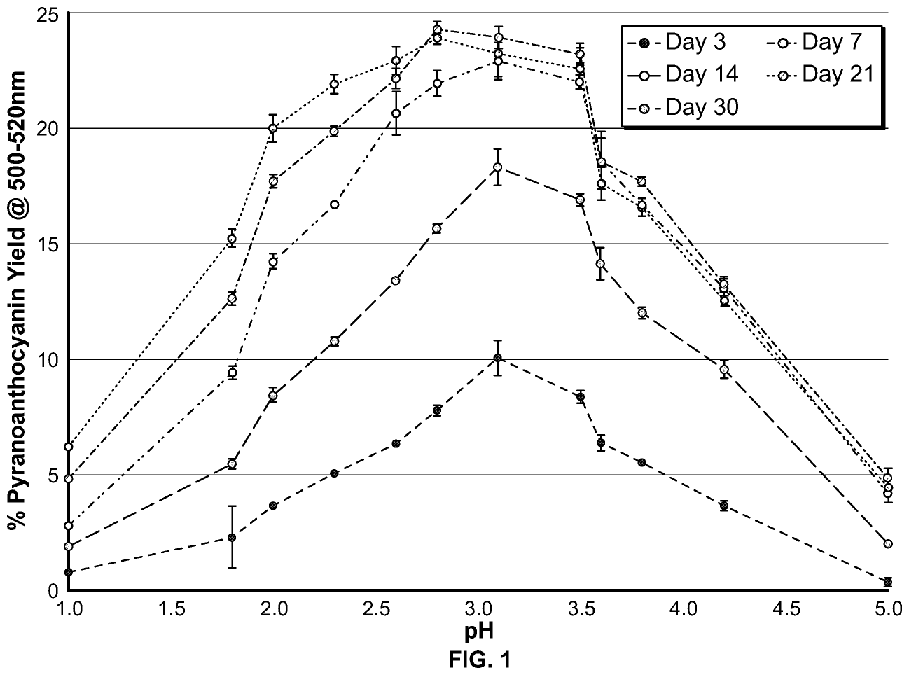

Pyranoanthocyanin formation yields were calculated in relation to the anthocyanin starting concentration on day 0. As shown in FIG. 1 pyranoanthocyanins were formed at all pH values, but at different yields. On day 7, a significantly greater pyranoanthocyanin formation yield (18.3%) was observed at pH 3.1 than any other pH value. On day 14, samples at pH 3.1 had the numerically largest pyranoanthocyanin formation yield at 23.0%, but this yield was not significantly larger than those of samples at pH 2.8 and pH 3.5. On day 21, samples at pH 2.8 (24.3%), 3.1 (23.9%) and 3.5 (23.2%) had the highest pyranoanthocyanin formation yields and were not significantly different from each other. Additionally, on day 30 pH 2.8 (23.9%), 3.1 (23.3%) and 3.5 (22.6%) were not significantly different from each other and day 30 yields were not significantly different from day 21 yields. On day 30 pH 2.6 reached similar yields to pH 2.8, 3.1 and 3.5 at 23.0%. It appears that a maximum yield of −24% under the conditions of this study could be obtained, as suggested by lack of significant difference between day 21 and 30. Reduced reaction time is preferable to obtain more pyranoanthocyanins in less time and to avoid anthocyanin and pyranoanthocyanin degradation and polymerization that further decreases pyranoanthocyanin yields (Rein et al., 2005). Importantly, the pyranoanthocyanin formation yields in this study were larger than previously recorded yields. For example, one study reported similar yields at 18.48% Vitisin A formation, but incubation took 232 days (Romero & Bakker, 2000) and another reported only 2.03% Vitisin A formation over 10 weeks (Murata et al., 2007).

In the first 7 days of the experiment pH 3.1 was the pH of maximum pyranoanthocyanin formation. Between days 14-21, formation began to slow and maximum yields were observed between pH 2.8 and pH 3.5 (FIG. 1). By day 30 maximum yields were observed between pH 2.6 and pH 3.5. The reported pK_{H} of cyanidin-3-glucoside is around 3.01, and the pK_{H} of blackberry has been reported as 3.07 (Stintzing, Stintzing, Carle, Frei, & Wrolstad, 2002), and both are near the maximum of pyranoanthocyanin formation at pH of 3.1. Pyranoanthocyanins form by nucleophilic substitution at the C-4 position of the anthocyanin molecule (Quaglieri et al., 2017). The anthocyanin flavylium cation has electrophilic character and the vinyl group of the cofactor (pyruvic acid in the enol form) has nucleophilic character allowing for the formation of pyranoanthocyanins (Dangles & Fenger, 2018). Pyranoanthocyanins likely form most efficiently when anthocyanins are predominantly in the flavylium cation form, because of the electrophilic character of the anthocyanin equilibrium state (Dangles & Fenger, 2018; Quaglieri, et al., 2017). However, if the only factor affecting formation was the need for the flavylium cation then optimum formation would have been near pH 1.0. This indicated pyruvic acid form and stability likely influenced formation. Pyruvic acid is known to undergo acid polymerization. As the pH decreases polymerization increases, so at low pH levels pyruvic acid polymerizes and becomes unavailable to react with the anthocyanin (Müller & Baumberger, 1939; Hazen, & Deamer, 2007). In addition, at more acidic pH values the keto form is favored over the enol form, so larger pH values encourage the enol equilibrium form (Müller & Baumberger, 1939). Therefore, it was predicted that the optimum pH in this study depended on more than one factor. The medium needed be acidic enough for the flavylium cation to be present in high quantities, but basic enough to prevent acid-catalyzed polymerization of pyruvic acid and basic enough to promote enolization.

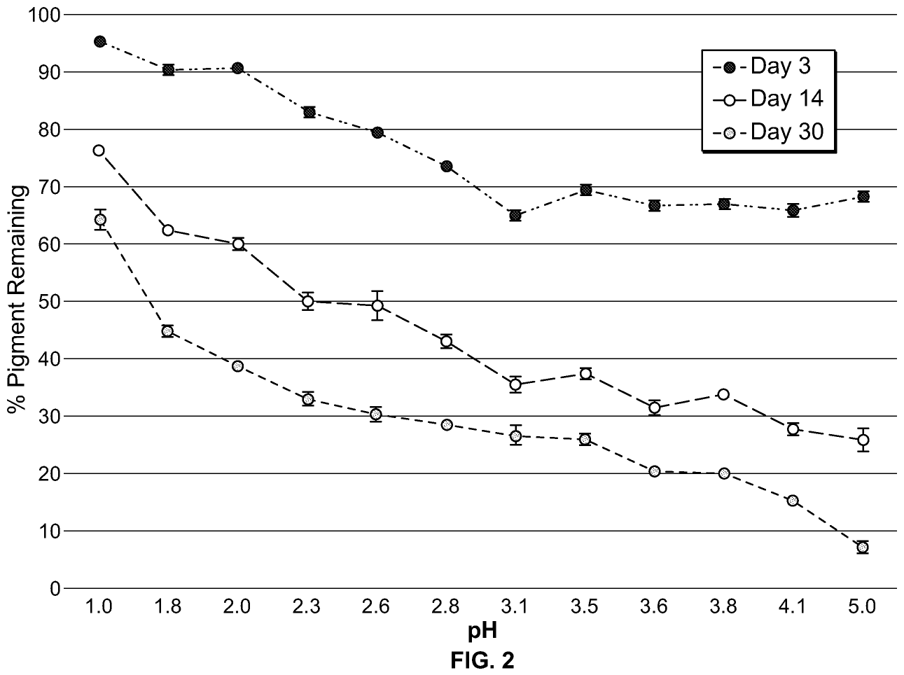

In the first 7 days of the experiment it was clear that pyranoanthocyanins were forming most rapidly at pH 3.1, indicating pH 3.1 was likely the preferred pH of formation in this study. As the study continued the non-significant difference for pyranoanthocyanin formation yields for pH 2.8, 3.1 and 3.5 showed surrounding pH values produced similar pyranoanthocyanin yields but at a slower rate of formation. Between days 14 and 21, there was a maximum formation between pH 2.8 and 3,5, and this pH range was slightly more acidic than traditional wine pH (pH 3.7-4.0) at which most pyranoanthocyanin formation studies have been carried out (Somers, 1971; Morata et al., 2007). Between pH 2.8 and 3.5, the flavylium cation was likely at higher concentrations in solution and near the pK_{H} of the anthocyanins. As the pH increased above 3.5 the colorless, neutral, hemiketal form began to predominate. As pH increased the anthocyanin molecules became more unstable, and began degrading, decreasing the number of anthocyanins present for the pyruvic acid to react with (FIG. 2).

### Spectrophotometric and Colorimetric Properties

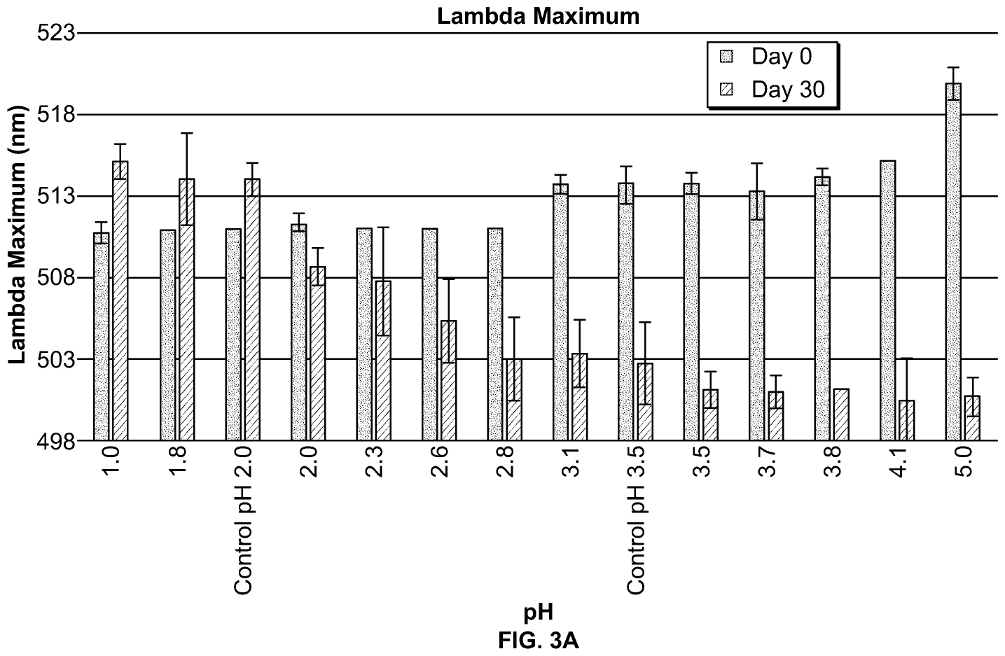

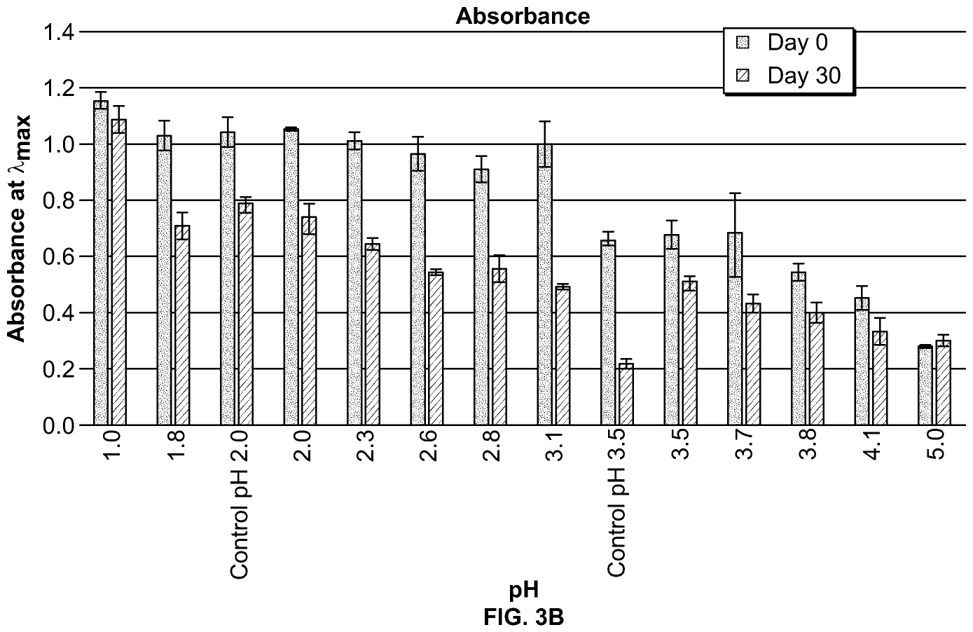

Samples showed a hypsochromic shift in the lambda maximum (λₘₐₓ) excluding pH 1.0 (significant λₘₐₓ increase) and 1.8 (not significant λₘₐₓ increase), FIG. 3. As shown in FIG. 3 the control sample at pH 2.0 followed a similar trend to treatment samples at pH 1.0 and 1.8 likely indicating the bathochromic shift at pH 1.0 and 1.8 occurred from anthocyanin reactions. Anthocyanins can undergo self-association and polymerization potentially leading to the bathochromic shifts (Brownmiller, Howard, & Prior, 2008; Fernandes, Brás, Mateus, & De Freitas, 2015). The degree of the hypsochromic shifts for the other samples (pH 2.0, 2.3, 2.5, 2.8, 3.1, 3.5, 3.7, 3.8, 4.1, 5.0) was likely attributed to pyranoanthocyanin formation and pigment degradation.

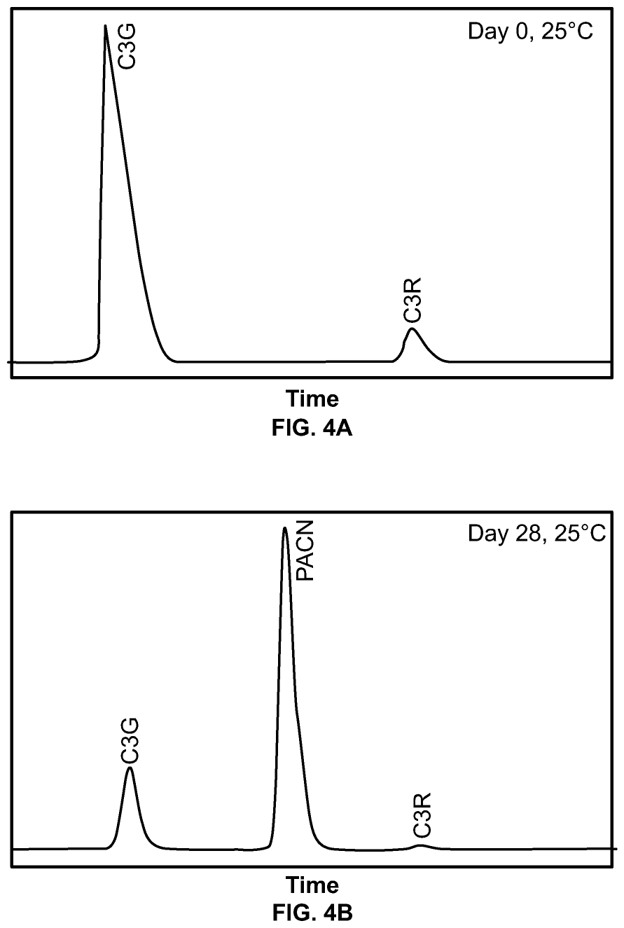

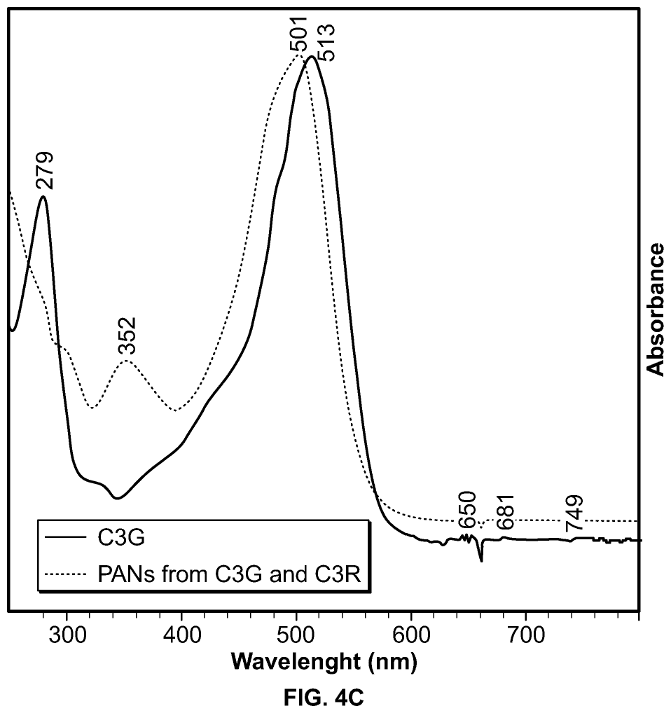

As shown in FIG. 4 pyranoanthocyanins are hypsochromicly shifted in the lambda maximum compared to their anthocyanin counter parts (De Freitas & Mateus, 2011; Oliveira et al., 2006). However, even with lesser amounts of pyranoanthocyanins formed at high pH values large hypsochromic shifts were still identified. FIG. 2 showed as pH increased a greater amount of pigment was degraded, so the large hypsochromic shifts at high pH values were likely attributed to pigment degradation. Shifts in the lambda maximum (λₘₐₓ) between day 0 and day 30 for samples with high pyranoanthocyanin formation, pH 2.8 (Δ8 nm), 3.1 (Δ10 nm) and 3.5 (Δ13 nm), can likely be attributed to both pyranoanthocyanin formation and pigment degradation. Samples at all pH values had a decrease in absorbance at the λₘₐₓ, consistent with loss of chromophores. FIG. 3 shows the pH 3.5 control sample had a significantly lower absorbance than the pH 3.5 treatment sample indicating the pyranoanthocyanins that formed may have helped protect the samples pigments.

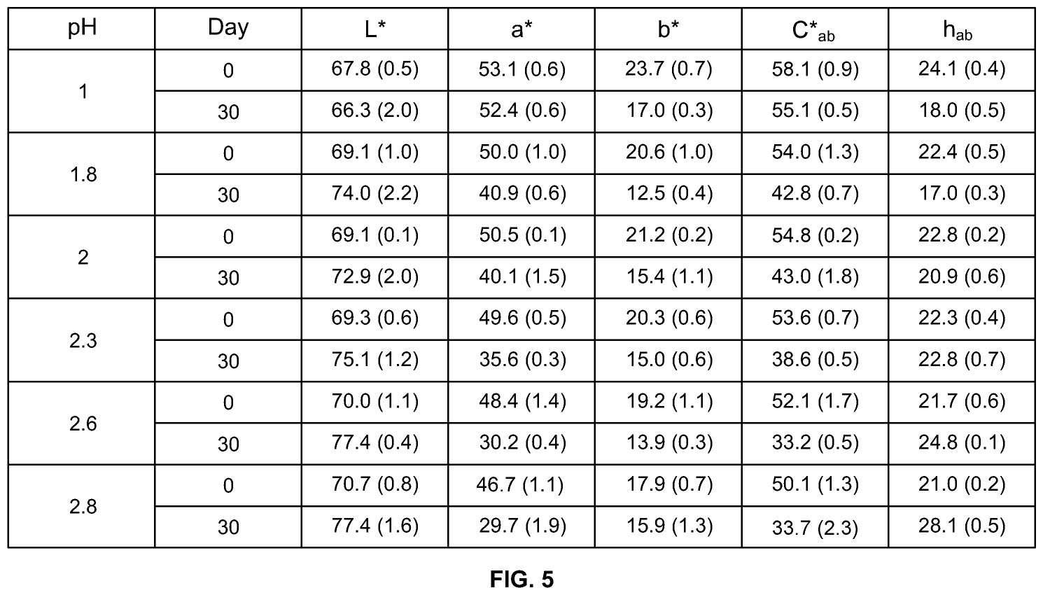

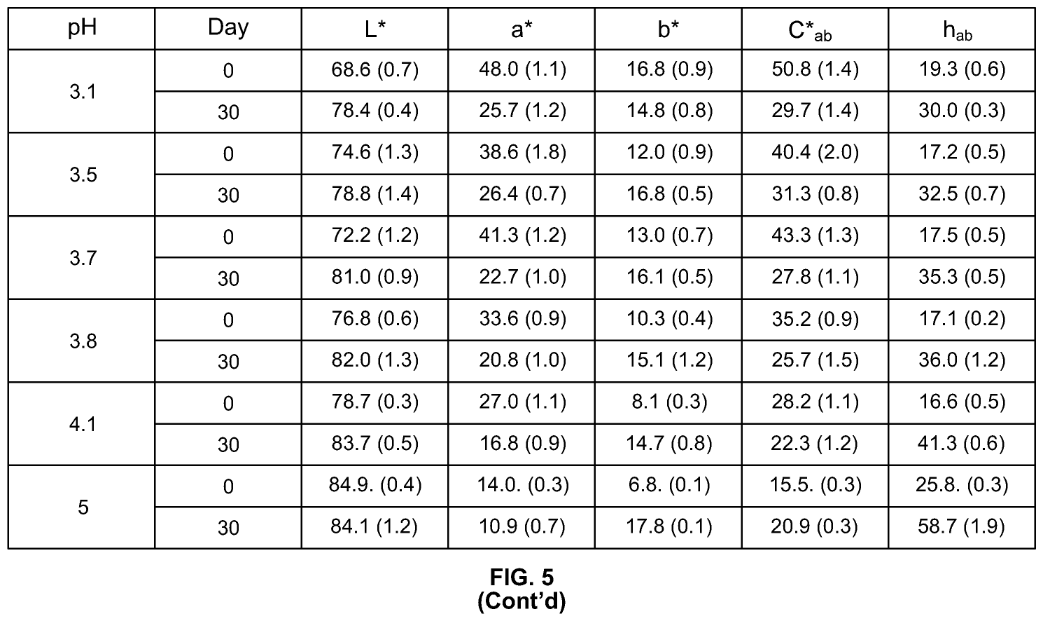

The color properties of all samples, on day 0 and day 30, are shown in FIG. 5. All samples showed an increase in lightness besides those at pH 1.0 and pH 5.0. At pH 1.0 anthocyanins are relatively stable; as shown in FIG. 2, sample at pH 1.0 had significantly more pigment remaining than any other sample which may have helped stabilize the L* value. At pH 5.0 the sample had a large L* value on day 0 and day 30, because the colorless hemiketal form of the anthocyanin predominates at pH 5.0. All samples showed a decrease in a*, meaning the color lost some redness. The b* decreased for all samples with pH values bellow 3.1 indicating samples became less yellow and with pH values greater than 3.1 the b* increased indicating samples became more yellow. The samples started out with a vibrant red color. As pyranoanthocyanins formed the samples became more orange in color, and as pigments degraded the samples became browner. The changes in a* and b* were likely attributed to both pyranoanthocyanin formation and pigment degradation (Rein et al., 2005; Wrolstad et al., 2005),

The chroma (C*_{ab}) decreased between day 0 and day 30 for samples at all pH values excluding pH 5.0. At pH 5.0 the colorless hemiketal predominated explaining the low initial chroma reading, and the formation of degradation products throughout the study may have increased the chroma. For other samples, chromophores were lost as pigments degraded, and the chroma decreased (He et al., 2012). The hue angle (h_{ab}) describes the shade of the color; for example, a h_{ab} of 0° is a red color and a h_{ab} of 90° is a yellow color. At pH 1.0, which had a pyranoanthocyanin formation yield of 6.3%, the hue angle decreased from 24.1° on day 0 to 18.0° on day 30. At pH 3.1, which had a pyranoanthocyanin formation yield of 23.9%, the h_{ab} increased from 19.3° (day 0) to 30.0° (day 30). Pyranoanthocyanins are more orange in color (De Freitas & Mateus, 2011; Oliveira et al., 2006) which likely attributed to the increase in h_{ab} (approaching 90°) for samples large pyranoanthocyanin formation yields. Additionally, as anthocyanins degrade the color changes from a red to a brown/yellow color. Therefore, the degradation of the anthocyanins could also have contributed to the shift in hue angle (Wrolstad, Durst, & Lee, 2005).

### Effect of Temperature on Pyranoanthocyanin Formation

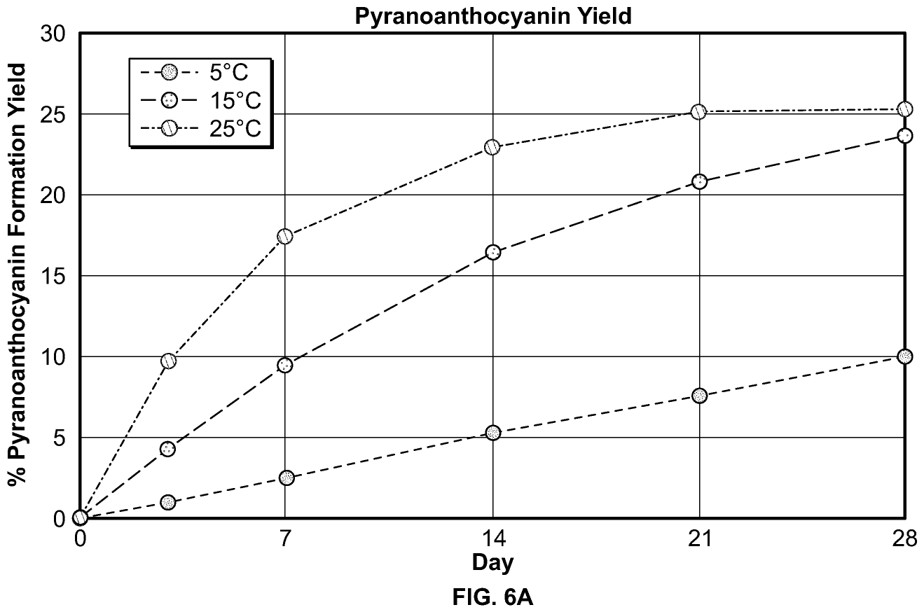

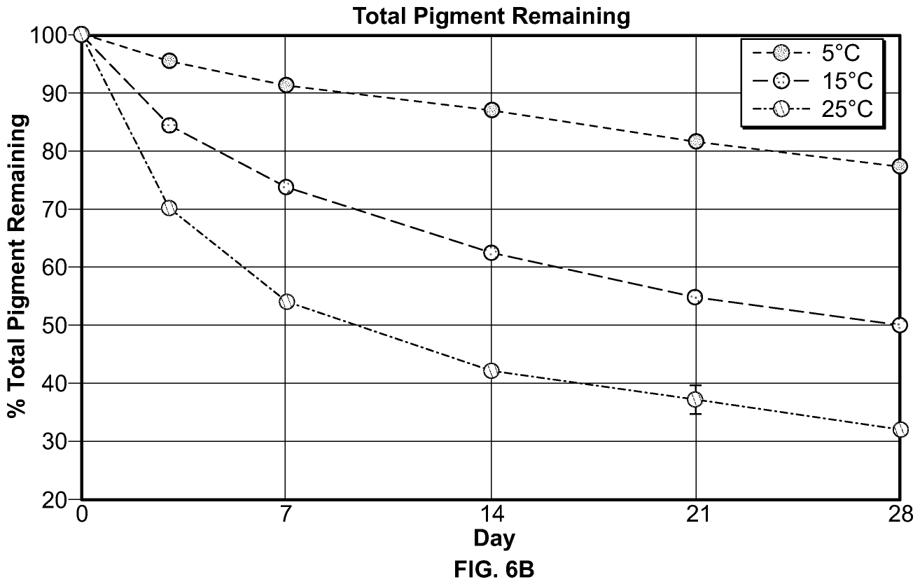

At all storage temperatures, pyranoanthocyanins were formed; however, there was a significant difference between pyranoanthocyanin formation yields for 5° C., 15° C., and 25° C. at each time point. On day 28. storage at 25° C. yielded the highest pyranoanthocyanin formation at 25.4%, followed by 15° C. at 23.7% and 5° C. at 10.0% formation (FIG. 6A). It was expected that as the temperature increased pyranoanthocyanin formation would increase. Collision theory explains that reactant partners must collide with a minimum energy (activation energy) to form a product. As the temperature of the system increases, molecules gain energy, increasing the number of collisions thus increasing the reaction rate (Brown et at, 2012). However, increased temperature also increased anthocyanin degradation (Eiro & Heinonen, 2002; Cabrita et al., 2000) as shown in FIG. 6 B. On day 28, at 25° C. 32.1% of the total pigments remained and at 15° C. 50.0% of the total pigments remained. Although storage at 25° C. yielded significantly more pyranoanthocyanin formation at all time points, it also led to significantly more pigment degradation at all time points.

There was no significant difference between pyranoanthocyanin formation yields on day 21 and day 28 when incubated at 25° C. However, a significant difference existed between the percentage of pigment remaining between day 21 and day 28 for 25° C. To efficiently form a compound, high yields should be obtained in the shortest amount of time. Therefore, 21 days was the preferred incubation time at 25° C. under the conditions of this study because pyranoanthocyanin formation was the same at 28 days but pigment degradation was lower on day 21.

### Spectrophotometric and Colorimetric Properties

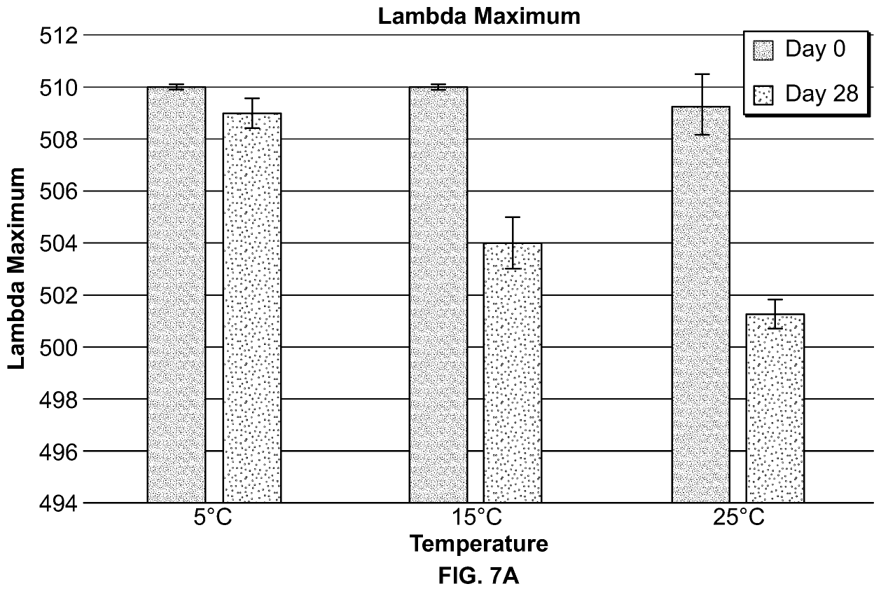

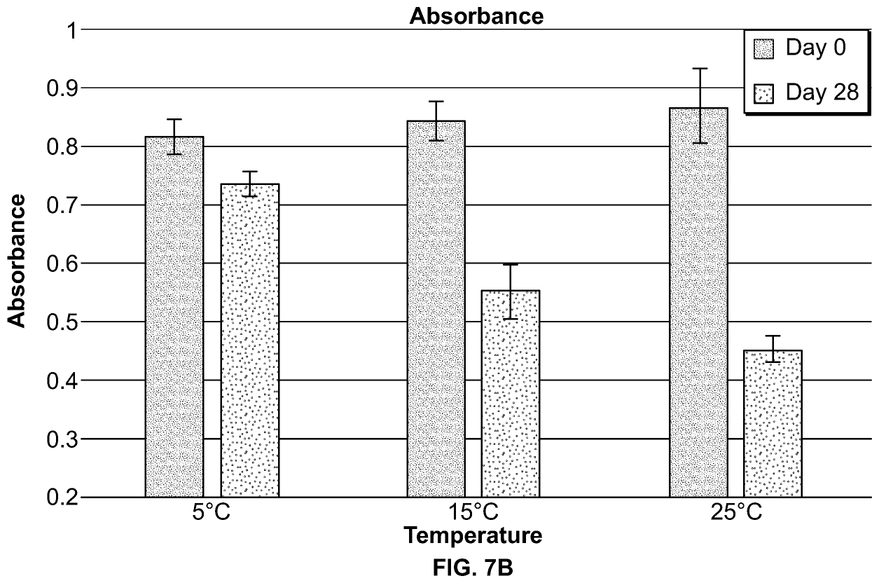

Spectral properties were recorded at each time point. As shown in FIG. 7 all samples had a decrease in the lambda maximum (λₘₐₓ) and a decrease in absorbance. The decrease in λₘₐₓ and absorbance were not significant at 5° C., because the low temperature likely helped stabilize the pigment. Pyranoanthocyanins had a hypsochromic shift in the λₘₐₓ in respect to the anthocyanin counterpart (FIG. 4), aligning with literature (Farr et al., 2018). Therefore, the hypsochromic shift in the λₘₐₓ between day 0 and day 28 was partially attributed to pyranoanthocyanin formation. In addition, pigments were degrading, likely contributing to the hypsochromic shift. Chromophores were lost as pigments degraded describing the decrease in absorbance over the 28 days.

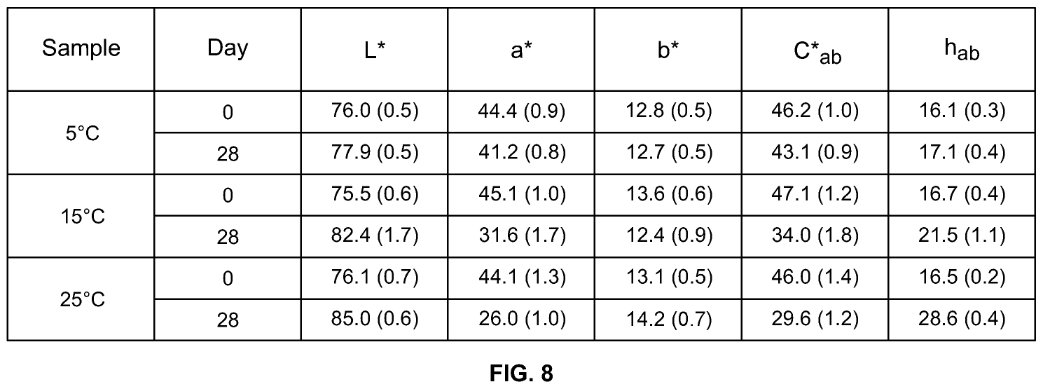

Color analyses were conducted and recorded at each time point. The color properties in FIG. 8 followed similar trends to the color properties in FIG. 5. L* increased, a* decreased, C*_{ab} decreased and h_{ab} increased. There was no significant difference in b* between day 0 and day 28 at any temperature. These variations can be described by both pyranoanthocyanin formation and pigment degradation, because pyranoanthocyanins are more orange in color compared to their anthocyanin counterpart and degraded pigments are browner in color (Rein et al., 2005; Wrolstad et al., 2005).

### Conclusion

This example focused on increasing pyranoanthocyanin yields by investigating effects of pH and incubation temperature. pH 3.1, which was near the PK_{H}, had the highest pyranoanthocyanin formation yields on day 7. Day 21 had an optimum pH range, pH 2.8 to 3.5, for pyranoanthocyanin formation yields (yields −24%), and day 21 was preferred over day 30. It was hypothesized that the pH of greatest formation depended on the pH being acidic enough for the flavylium cation form to predominated, but basic enough to discourage pyruvic acid polymerization and encourage enol formation. Therefore, the pH range of greatest formation was more acidic than typical wine pH but not too acidic. 25° C. had the highest pyranoanthocyanin formation yields by day 21 (yields ˜25%). As temperature decreased pyranoanthocyanin formation took more time and yields decreased, because the energy in the system decreased.

### Example 3. Effect of Flight Energy on Photoisomerization of acylated anthocyanin for the Expansion of Their Color Expression

Delphinidin-3-(p-coumaroyl)-rutinoside-5-glucoside was extracted from Chinese eggplants, semi-purified and taken to 400 mg/L in methanol. Extract aliquots were placed in sealed quartz cuvettes, with the clear side facing up, and irradiated with different light sources (sunlight, D₆₅ lamp, and UV chamber at 365 nm) for up to 112 hrs. Pigment isomerization was monitored over time on a uHPLC-PDA-MS/MS, using a pentafluorophenyl column and positive electrospray ionization on a triple quadrupole. Cis and trans-delphinidin-derivatives were isolated to compare their color stability and strength. Color was measured on a ColorQuest XE spectrophotometer, using 10° observer angle, illuminant D65, and total transmittance. Data was collected in triplicates and analyzed by two-way ANOVA.

All of the light treatments induced the trans- to cis-delphinidin conversion. However, greatest trans to cis-delphinidin conversion occurred with minimal total pigment degradation when the extract was irradiated for 10 hours with D65 lamp, Sunlight yielded less total ACN degradation, but the isomerization reaction was slow (>4 days). Isomerization occurred within a few hours in the UV chamber at 365 nm, but with high degradation. Color expression of the two configurations differed according to the pH. In pH 4, cis-delphinidin exhibited noticeable color, while its trans-counterpart bleached completely. In pH 5, cis- showed greater color intensity than the trans-isomer. In pH 7, the two isomers exhibited two different hues.

### Example 4. Improving Colorimetric Properties and Stability of acylated anthocyanins through UV Irradiation

Cyanidin-derivatives were extracted from American elderberry fruits. Cis- and trans-coumaroylated Cy-3-sambubioside-5-glucoside were isolated by semi-preparative HPLC. Crude extracts and purified trans-isolates with anthocyanin concentration from 100 μM to 800 μM were irradiated by UV light (254 nm) in acidified (0.1% HCl) aqueous and alcoholic (methanol/ethanol) solution until a plateau was reached. Every 2 min, 100 μl aliquots were injected into μHPLC-MS to monitor the conversion rate. UV-irradiated crude extract, purified cis- or trans-isomers were diluted to 100 μM in pH 1-9 buffers and spectrophotometric and colorimetric properties were collected during 72 h of storage using a UV-Vis plate reader.

The configuration of coumaric acid affected anthocyanin spectral and stability properties. Cis-isomers exhibited sharper spectra, larger λₘₐₓ and absorbance under all pH, showing less bleaching under pH 4-6 and bluer hues under pH 7-9. Cis-isomers also exhibited improved stability under all pH. Trans→cis conversion happened in both trans-isolates and elderberry crude extract, with similar conversion efficiency. UV light triggered the trans→cis conversion in both alcoholic and aqueous solution, and reached to the plateau in 16 minutes, with the trans-to-cis ratio of 5-to-4 in alcoholic solution and 5-to-3 in aqueous solution. Though the trans→cis conversion favored under lower anthocyanin concentration, the production of cis-isomers increased with anthocyanins concentration. The elderberry crude extract in aqueous solution after UV irradiation, showed a much bluer hue at neutral and alkaline condition.

### Example 5. Improving anthocyanin Color Performance with Efficient pyranoanthocyanin Formation

Pyranoanthocyanins, first found in wine, are a naturally derived pigment formed from anthocyanins and a cofactor over time. Anthocyanins are used in the food industry as a natural colorant but degrade quickly, causing them to lose color. Pyranoanthocyanins are more stable but form inefficiently. The present example describes a method of producing a significant concentration of pyranoanthocyanins within a day by increasing incubation temperature. Comparatively, previous research has shown comparable pyranoanthocyanins formation at 42 days. The method described in this example can save the food industry a significant amount of time and money in producing pyranoanthocyanins to use in food products as a natural colorant. Additionally, this method makes pyranoanthocyanins more applicable as a food colorant for industry by increasing the amount of pigment that can be produced over time.

Food colorant toxicity has long been a matter of concern for consumers and regulatory bodies. In a double-blinded, randomized trial, children consuming food with artificial colors had increased hyperactivity (see McCann, D. et al. ‘Food additives and hyperactive behavior in children community.’ Lancet 2007, 370:1560-67). Anthocyanins (ACN) are plant pigments that can serve as natural food colorants but are unstable. ACN are also powerful antioxidants and may promote health. Pyranoanthocyanins (PACN), naturally formed in red wine from CAN, are more stable but are formed inefficiently (see Rentzsch et al. ‘Pyranoanthocyanins: an overview” Trends Food Sci Technol 2007, 18 (10):526-534).

The studies outlined in the present example explored whether PACN could be efficiently produced using caffeic acid (CA) as a cofactor and with temperature as a catalyzer. To this end, semi-purified elderberry ACN were mixed with CA (1:50 ACN:CA) in pH 3 buffer. The effect of temperature was tested in samples with or without CA incubated at 25, 50, or 75° C. for 7 days. Different ACN/CA molar ratios (1:0, 1:50, 1:100, 1:150, 1:200) were tested at 65° C. for 8 days. PACN formation was monitored on an uHPLC-PDA-MS/MS.

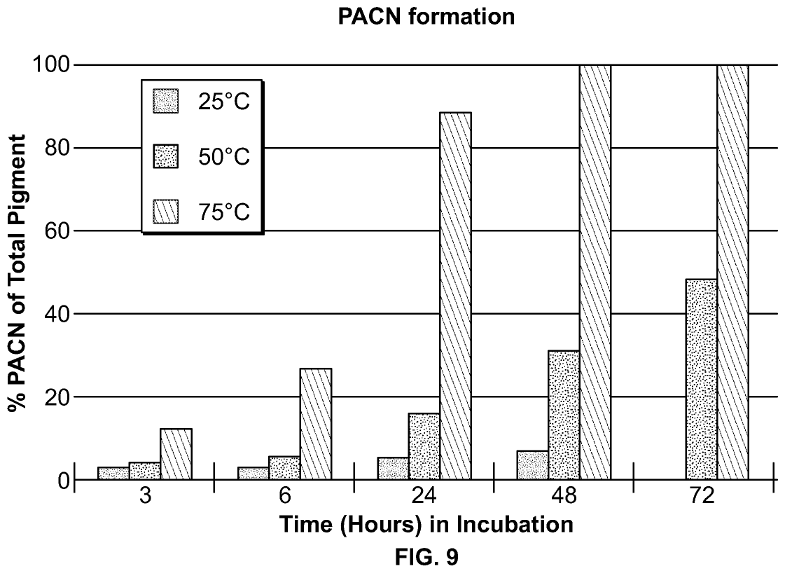

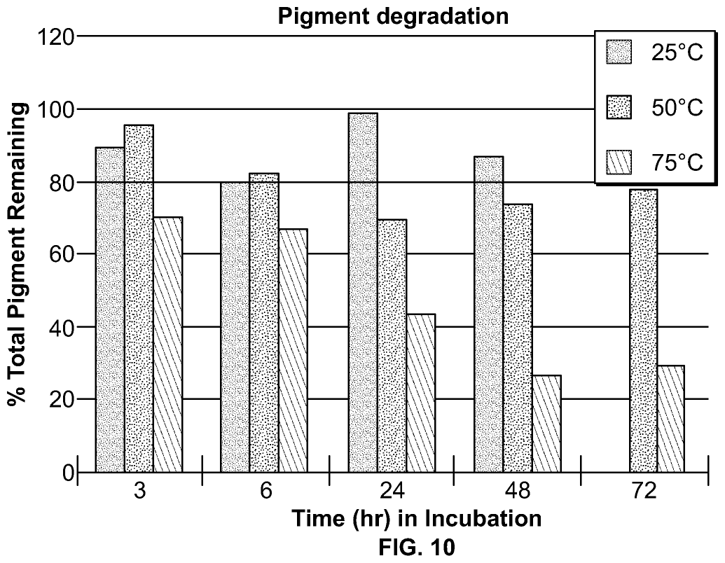

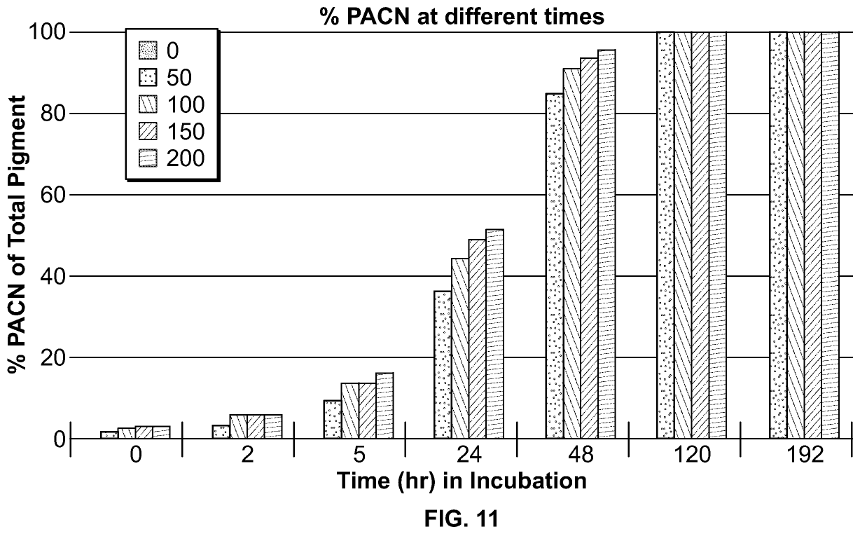

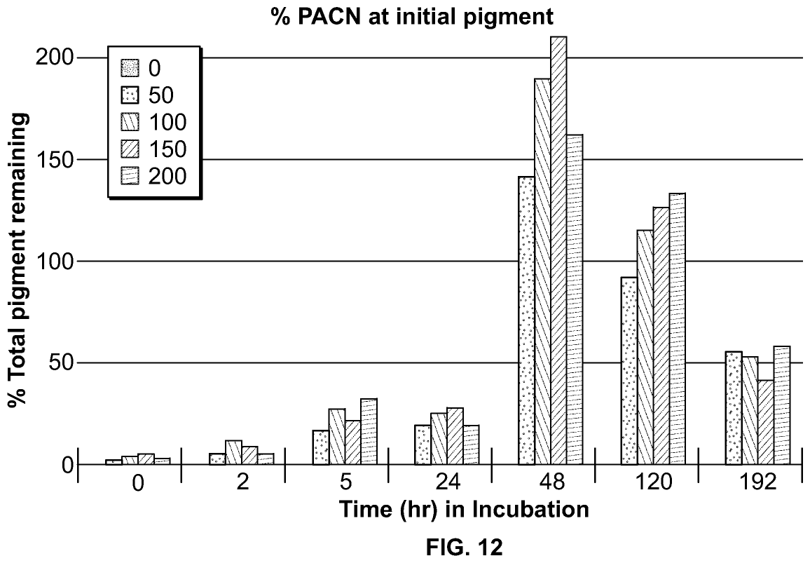

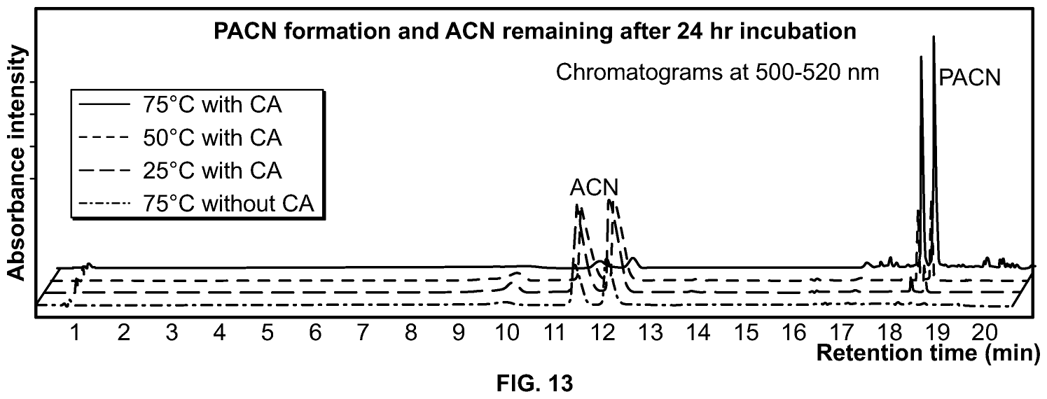

PACN was found to form faster at higher temperatures (see FIG. 9), with 100% of ACN converted to PACN within 2 days. PACN levels reached at 75° C. in 24 hours took 42 days to form at 25° C. Total pigment content decreased more rapidly at higher temperatures (see FIG. 10). Higher ACN:CA ratios produced PACN more rapidly (see FIG. 11). An ACN:CA molar ratio of 1:150 had the greatest PACN formation, reaching 212% of the initial pigment absorbance (see FIG. 12). Higher temperatures lead to lower ACN and higher PACN levels after 24 hours (see FIG. 13). In conclusion, elderberry ACN can be combined with CA and heat to efficiently produce the more stable PACN with potential application as naturally derived food dyes as alternatives to artificial dyes.

### Example 6. Monitoring Hydroxycinnamic Add Decarboxylation by Lactic Acid Bacteria Using High-Throughout UV-Vis Spectroscopy

Hydroxycinnamic acids (HCAs) are secondary metabolites in plants and fungi characterized by a C6-C3 structure (Pereira, D. M.; Valentão, P.; Pereira, J. A.; Andrade, P. B. Phenolics: From chemistry to biology. Molecules 2009, 14, 2202-2211). At high concentrations, these compounds exert an antibacterial effect over a wide range of Gram-positive and Gram-negative bacteria (Herald, P. J.; Davidson, P. M. Antibacterial activity of selected hydroxycinnamic acids. J. Food Sci. 1983, 48, 1378-1379; Borges, A.; Ferreira, C.; Saavedra, M. J.; Simões, M. Antibacterial activity and mode of action of ferulic and gallic acids against pathogenic bacteria. Microb. Drug Resist, 2013, 19, 256-265; and Lou, Z.; Wang, H.; Rao, S.; Sun, J.; Ma, C.; Li, J. p-Coumaric acid kills bacteria through dual damage mechanisms. Food Control 2012, 25, 550-554), Further studies have shown that some bacterial strains can decarboxylate HCAs as a mechanism for detoxification of its environment, given that the antimicrobial activity of the aforementioned acids depends on the presence of a double bond in the side chain of the structure (Sanchez-Maldonado, A. F.; Schieber, A.; Ganzle, M. G. relationships of the antibacterial activity of phenolic acids and their metabolism by lactic acid bacteria. J. Appl. Microbiol. 2011, 111, 1176-1184). However, it also has been reported that not all bacterial (Couto, J. A.; Campos, F. M.; Figueiredo, A. R.; Hogg, T. A. Ability of lactic acid bacteria to produce volatile phenols. Am. J. Enol. Vitic. 2006, 57, 166-171) or all yeast strains (Shinohara, T.; Kubodera, S.; Yanagida., F. Distribution of phenolic yeasts and production of phenolic o_-flavors in wine fermentation. J. Biosci. 2000, 90, 90-97) are capable of decarboxylating HCAs that lead to the formation of 4-vinylphenols (4VPs) or their reduced form, 4-ethylphenols. In fermented products such as wine, HCA degradation by yeast results in the formation of 4VPs; which are responsible for imparting distinct phenolic off-flavors in the final product. However, depending on the application, these compounds may be desirable for cases such as wheat beer, where the reduction in 4VP and 4-vinylguaiacol (4VG) production resulted in a less pronounced wheat beer aroma (Langos, D.; Granvogl, M. Studies on the simultaneous formation of aroma-active and toxicologically relevant vinyl aromatics from free phenolic acids during wheat beer brewing. J. Agric. Food Chem. 2016, 64, 2325-2332); or artisanal bread, where decarboxylation of ferulic acid (FA) generated 4VG, an important component in its flavor profile (Wang, H. E.; Hwang, C. F.; Tzeng, Y. M.; Hwang, W. Z.; Mau, J. L. Quality of white bread made from lactic acid bacteria-enriched dough. J. Food Process, Preserv. 2012, 36, 553-559). In addition, 4VPs do not only affect the flavor of foods, but also their color, as they can react with other components in the food matrix such as anthocyanins. This interaction can result in the formation of pyranoanthocyanins (Benito, S.; Morata, A.; Palomero, F.; Gonzalez, M. C.; Suarez-Lepe, J. A. Formation of vinylphenolic pyranoanthocyanins by Saccharomyces cerevisiae and Pichia guilllermondii in red wines produced following different fermentation strategies. Food Chem. 2011, 124, 15-23), compounds partially responsible for imparting color to aged red wines with an enhanced resistance to bleaching (Farr, J. E.; Giusti, M. M. Investigating the interaction of ascorbic acid with anthocyanins and pyranoanthocyanins. Molecules 2018, 23. 744; and De Freitas, V; Mateus, N. Formation of pyranoanthocyanins in red wines: A new and diverse class of anthocyanin derivatives. Anal. Bioanal. Chem. 2011, 401, 1467-1477). In dairy foods, HCA decarboxylation by LAB is of special importance in cheese and yogurt, as nowadays it is not uncommon to find these products fortified with HCA-rich plant extracts (Oh, N. S.; Lee, J. Y.; Joung, J. Y.; Kim, K. S.; Shin, Y. K.; Lee, K. W.; Kim, S. H.; Oh, S.; Kim, Y. Microbiological characterization and functionality of set-type yogurt fermented with potential prebiotic substrates Cudrania tricuspidata and Morus alba L. leaf extracts. J. Dairy Sci, 2016, 99, 6014-6025; Ruggeri, S.; Straniero, R.; Pacifico, S.; Aguzzi, A.; Virgili, F. French marine bark extract pycnogenol as a possible enrichment ingredient for yogurt. J. Dairy Sci. 2008, 91, 4484-4491; and Khan, I. T.; Nadeem, M.; Imran, M.; Ajmal, M.; Ali, S. Antioxidant activity, fatty acids characterization and oxidative stability of Gouda cheese fortified with mango (Mangifera indica L.) kernel fat. J. Food Sci. Technol. 2018, 55, 992-1002). Moreover, the increasing use of pigment-rich plant extracts as food colorants can also result in an increased HCA content (Karaaslan, M.; Ozden, M.; Vardin, H.; Turkoglu, H. Phenolic fortification of yogurt using grape and callus extracts. Lwt Food Sci. Technol. 2011, 44, 1065-1072; and Coïsson, J. D.; Travaglia, F.; Piana, G.; Capasso, M.; Arlorio, M. Euterpe oleracea juice as a functional pigment for yogurt. Food Res. Int. 2005, 38, 893-897). Depending on the LAB strain used during production, these HCAs could he biotransformed into 4VPs, affecting the sensorial characteristics of the final product. Therefore, the study of the decarboxylating capabilities of LAB strains isolated from dairy products is a subject of increasing interest in the food industry. The most common methodologies for the screening of these decarboxylated products have been HPLC-DAD-MS, GC-FID (Curiel, J. A.: .Rodriguez, H.; Landete, J. M.; de las Rivas, B.; Munoz, R. Ability of Lactobacillus brevis strains to degrade food phenolic acids. Food Chem. 2010, 120, 225-229) or GC-MS (Thurston, P. A.; Tubb, R. S. Screening yeast strains for their ability to produce phenolic o_ -flavours: A simple method for determining phenols in wort and beer. J. Inst. Brew. 1981, 87, 177-179). These methodologies, although precise and reliable, require expensive equipment, trained personnel, and high costs of operation. A simpler spectrophotometric method for the detection of 4VPs in a mixture was first reported using quartz cuvettes and pH 6.0 buffer (Cavin, J. F.; Andioc, V.; Etievant, X.; Diviès, C. Ability of wine lactic acid bacteria to metabolize phenol carboxylic acids. Am. J. Enol. Vitic. 1993, 44, 76-80), showing hypsochromic shifts in the λₘₐₓ from 285-300 nm for HCAs to 260 nm for 4VPs. This hypsochromic shift can be the basis for a modified high-throughout method that could aid in the screening and selection of LAB capable—or incapable, depending on the application—of decarboxylating HCAs. These bacteria could be used to modulate the release of compounds that affect the sensory characteristics of fermented products elaborated from HCA-rich sources.

The objective of this example was to develop a fast and simple methodology for high-throughout screening of LAB that are capable of HCA biotransformation, in order to determine the decarboxylating activity of LAB strains.

### Results and Discussion

### UV-Vis Spectra of HCAs, Decarboxylated HCAs and Growth Medium

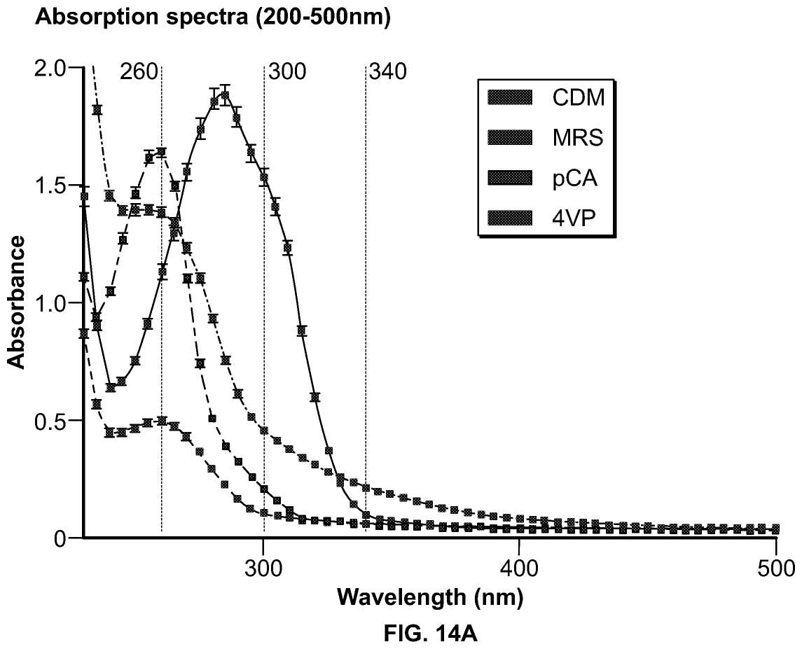

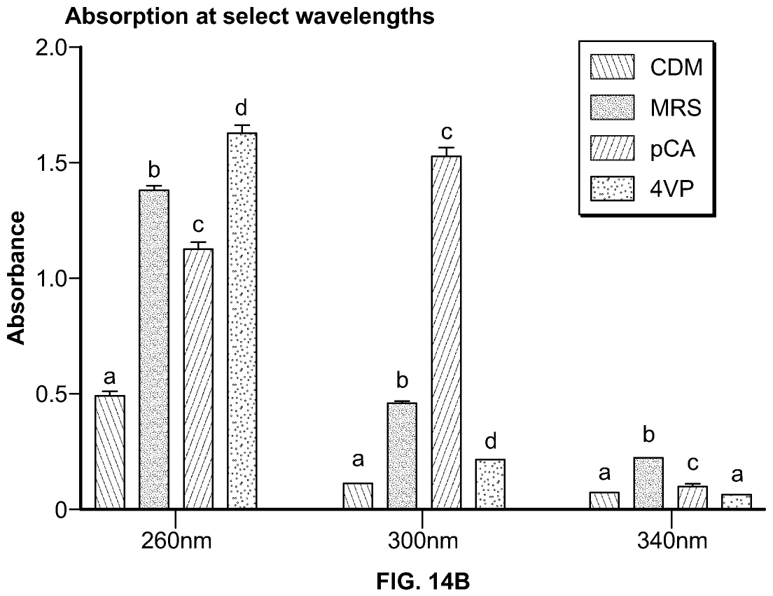

The first goal was to monitor the decarboxylation of HCAs using a fast, high-throughout method based on UV-Vis light absorption changes. FIG. 14A shows the UV-Vis light absorption spectra of p-coumaric acid (pCA), 4VP, a chemically defined medium (CDM), and de Man, Rogosa and Sharpe broth (MRS) all at pH 7.0. The wavelength of maximum absorption for pCA is near 290 nm (blue line), while the one registered for 4VP is near 260 nm (red line). These results are consistent with previously reported values for chromophores with similar structural characteristics (200-280 nm for phenolic acids and 300-350 nm for HCAs) (Robbins, R. J. Phenolic acids in foods: An overview of analytical methodology. J. Agric. Food Chem. 2003, 51. 2866-2887). Therefore, this UV-Vis light wavelength range (200-350 nm) was a critical factor for the selection of a culture medium in which the decarboxylation experiments could be conducted, and that will allow a direct measurement of UV-Vis light absorption changes in response to structural modifications in the chromophore after incubation with LAB. FIG. 14B shows the absorption of all samples at 260, 300 and 340 nm. Results showed that CDM (green bar) had a significantly lower absorption than MRS broth (black bar) at all three wavelengths analyzed (p<0.01). This significantly lower absorption makes CDM an ideal medium to conduct decarboxylation experiments due to a much lower interference with the UV-Vis light absorption of the compounds of interest. The significantly lower absorption of CDM in the UV-Vis wavelength range of interest when compared to MRS broth can be attributed to a lower concentration of protein-rich components in its formulation (5 g/L in CDM and 25 g/L in MRS). Therefore, MRS broth was used for initial cell growth until the log phase was reached, and CDM was used for decarboxylation experiments and subsequent UV-Vis light absorption measurements.

### Determination of the Optimal pH for Monitoring HCA Decarboxylation

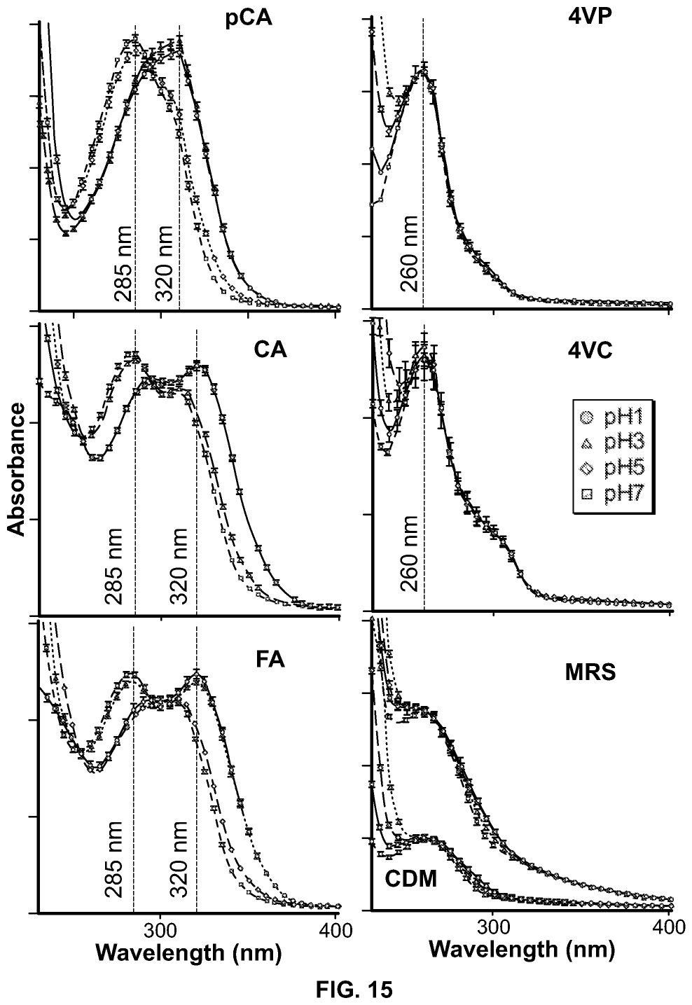

After selecting an appropriate culture medium to conduct decarboxylation experiments, the next step was the determination of the optimal pH for monitoring changes in the UV-Vis light absorption in response to HCA decarboxylation by LAB. It was previously noticed that absorption increased significantly in wavelengths shorter than 230 nm, and no valuable information was Obtained from that region of the spectra due to the high interference of the microplate used for assays. Therefore, subsequent experiments were focused on the light absorption changes in wavelengths longer than 230 nm. To determine the best pH for detection of changes in light absorption after incubation with LAB, the spectra of CDM containing HCAs or 4VPs were measured at pH 1.0, pH 3.0, pH 5.0 and pH 7.0. As shown in FIG. 15, the λ_{230-500 max} of HCAs registered a bathochromic shift when pH decreased. At pH 1.0 (red line) and 3.0 (orange line), the λ_{230-500 max} was 309 nm for pCA, 320 nm for caffeic acid (CA), and 320 nm for FA. At pH 5.0 (light green line) and 7.0 (green line), the absorption reading was 285 nm for pCA, 284 nm for CA, and 283 nm for FA. The similarity between the λ_{230-500 max} of acidic pH (pH 1.0 and pH 3.0) and near-neutral pH (pH 5.0 and pH 7.0) can be explained by the pKa of HCAs, which were reported to be between 4.0 and 4.5 (Ong, B. Y.; Nagel, C. W. High-pressure liquid chromatographic analysis of hydroxycinnamic acid-tartaric acid esters and their glucose esters in vitis vinifera. J. Chromatogr. A 1978, 157, 345-355; Erdemgil, F. Z.; ,Sanli, S.; ,Sanli, N.; Özkan, G.; Barbosa, J.; Guiteras, J.; Beltrán, J. L. Determination of pKa values of some hydroxylated benzoic acids in methanol-water binary mixtures by LC methodology and potentiometry. Talanta 2007, 72, 489-496; and Sanli, N.; Fonrodona, G.; Barron, D.; Özkan, G.; Barbosa, J. Prediction of chromatographic retention, pKa values and optimization of the separation of polyphenolic acids in strawberries. J. Chromatogr. A 2002, 975, 299-309). Therefore, at pH values lower than 4.0, the acid is expected to be in its undissociated form, while at pH values larger than the pKa, they are expected to be in their dissociated form, which ultimately changes the light absorption pattern of the chromophore. No major changes in the λ-500 max of decarboxylated products were observed in response to changes in the pH. Further measurements were conducted at pH 1.0 because it provided a more distinguishable hypsochromic shift between the λ_{230-500 max} of the HCAs and their decarboxylated products than buffers at pH 5.0 and pH 7.0. Although measurements in acidic pH (pH 1.0 and 3.0) showed similar absorbance values for the λ_{230-500 max}, pH 1.0 buffer was selected due to the lower absorbance of the medium at wavelengths below 260 nm. This provided a more defined peak in the spectra, thereby allowing for a better visualization of hypsochromic changes that occur as a result of HCA decarboxylation. Moreover, the light absorption of MRS broth and CDM at different pH values were also monitored. No significant differences were found in light absorption as a response to pH changes (p>0.05, data not shown); however, it is clear that the absorption of MRS broth is higher than that of CDM at all four pH values. With the data obtained, we proceeded to initially grow bacteria at their optimal pH in MRS broth, conducted decarboxylation experiments at their optimal pH in CDM, and diluted an aliquot 25 times with pH 1.0 buffer prior to measurements.

### HPLC-DAD-ESI-MS Analysis of Decarboxylated Products

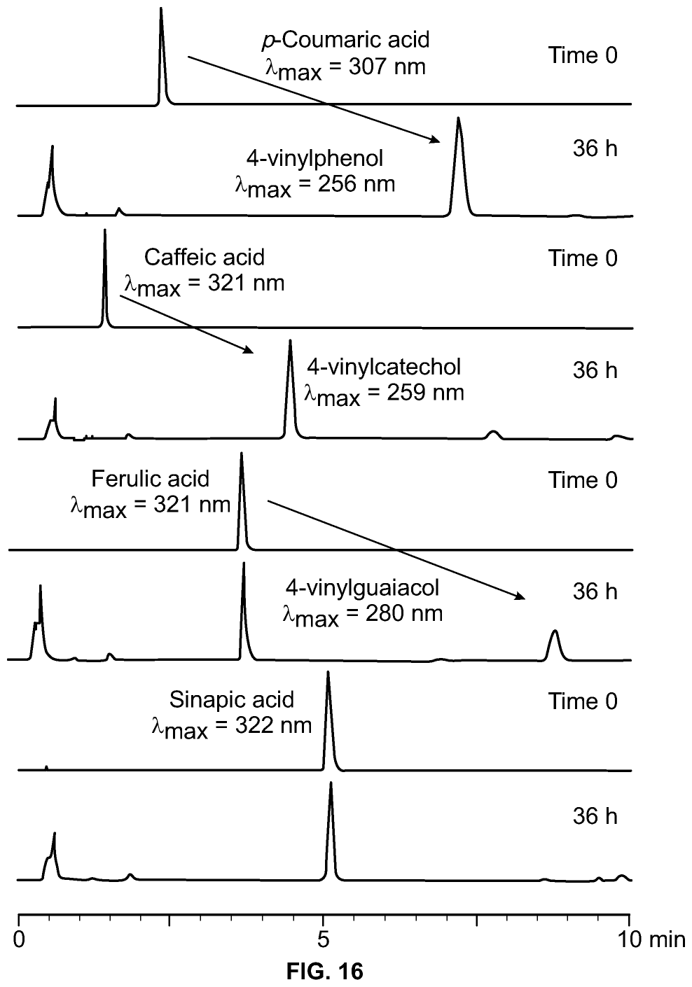

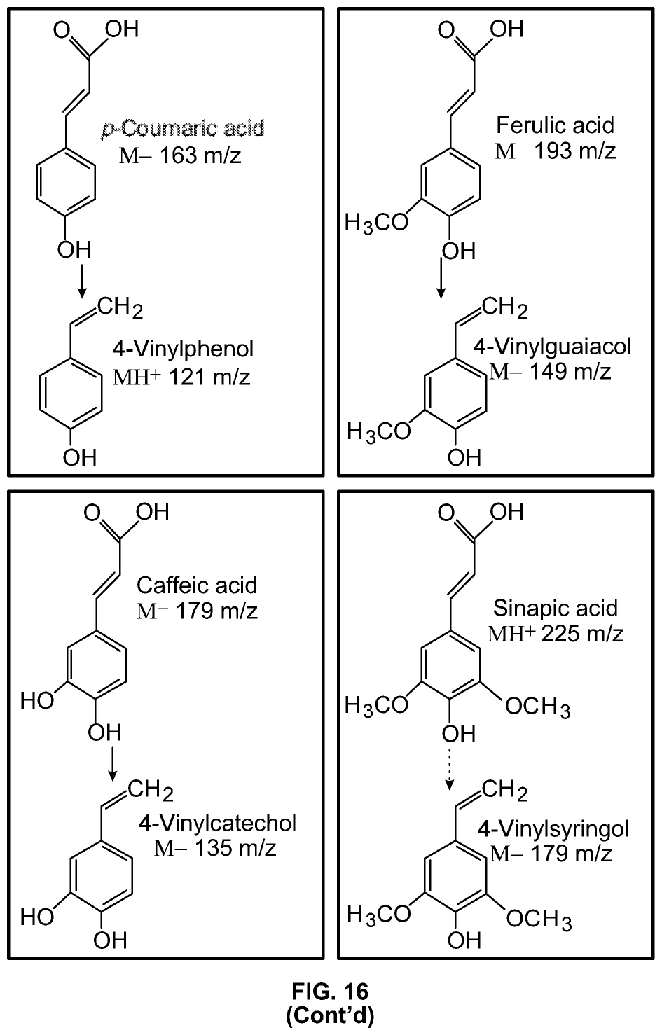

Results in FIG. 16 showed that the degradation products from pCA, CA and FA had longer retention times, and the absorption spectra of the product peak registered by the DAD, showed a hypsochromic shift in the λ_{230-500 max} when compared to the precursor HCA (>50 nm shift), consistent with the production of decarboxylated products (De Las Rivas, B.; Rodriguez, H.; Curiel, J. A.; Landete, J. M.; Mu{tilde under (n)}oz, R. Molecular screening of wine lactic acid bacteria degrading hydroxycinnamic acids. J. Agric. Food Chem. 2009, 57, 490-494). Under our experimental conditions, no sinapic acid (SA) decarboxylation was observed after incubation with LAB. This was denoted by the absence of peaks corresponding to common products from SA decarboxylation such as 4-vinylsyringol or 4-ethlysyringol. To better identify decarboxylated products, mass spectrometry analyses were conducted at time 0 and after 36 h of incubation at 32° C. in the dark, After incubation, HPLC-DAD-ESI-MS analyses confirmed the absence of pCA (M⁻ 163 m/z) and CA (M⁻ 197 m/z), and showed their transformation into their corresponding decarboxylated vinyl derivatives, 4-VP (MH⁺ 121 m/z) and 4-vinylcatechol (4VC, M⁻ 135 m/z), respectively. The product from the partial degradation of FA (M⁻ 193 m/z) was consistent with that of 4VG (M⁻ 149 m/z). Previous studies have reported the activity of a vinylphenol reductase resulting in the appearance of 4-ethylphenols derivatives from HCAs Fras, P.; Campos, F. M.; Flogg, T.; Couto, J. A. Production of volatile phenols by Lactobacillus plantarum in wine conditions. Biotechnol. Lett. 2014, 36, 281-285) However, in this example, the HPLC-DAD-ESI-MS analyses showed no evidence of 4-ethylphenol production. The 36-h incubation at 32° C. allowed for complete decarboxylation of both pCA and CA into their 4VP derivatives with no 4-ethylphenols, making them ideal substrates for monitoring the activity of phenolic acid decarboxylase in this proposed methodology. The characteristic m/z of decarboxylated products from SA (225 m/z), 4-vinylsyringol (179 m/z) and 4-ethylsyringol (181 m/z) were also monitored, but no signal was found by the MS detector.

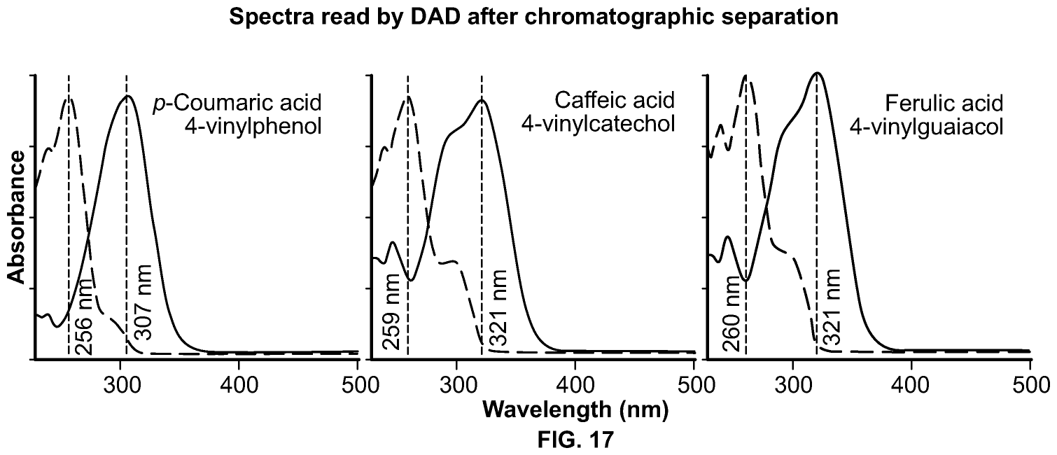

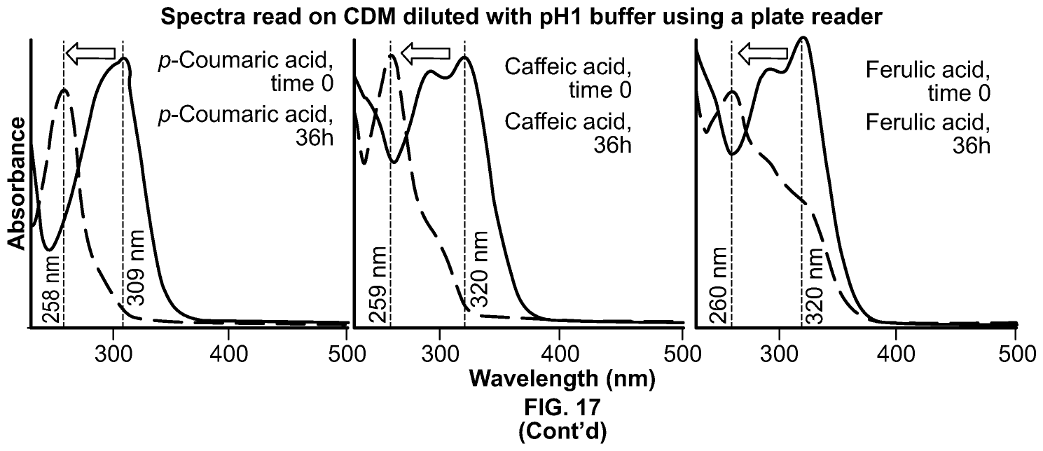

In FIG. 17, the absorption spectra of the individual peaks for each HCA and its respective decarboxylated derivatives (upper) are compared to the UV-Vis absorption of the bacterial supernatant measured with a plate reader directly after dilution (lower). Both methodologies were able to consistently detect hypsochromic shifts in pCA, CA and FA after incubation with LAB. These shifts occurred due to a decrease in the number of conjugated double bonds in the chromophore, as decarboxylation results in the loss of a double bond in the side chain of the HCA structure. This is consistent with previous literature showing the formation of decarboxylated products by phenolic acid decarboxylase. With the plate reader, the incubated samples displayed hypsochromic shifts of 51 nm for pCA, 61 nm for CA, and 60 nm for FA. These hypsochromic shifts were similar to the ones registered by the HPLC-DAD.

### Screening of LAB for Their Ability to Decarboxylate HCAs

LAB strains isolated from dairy products were screened for their ability to decarboxylase HCAs using the high-throughout UV-Vis spectrophotometric method described herein. Results from the initial bioinformatic analysis, showed that 22 strains from a total of 137 were potentially capable of synthesizing PAD based on their reported genome annotations, Lactobacillus plantarum was used as a positive control for PAD activity, and Staphylococcus epidermidis and Lactobacillus casei were used as negative controls. A total of 24 bacterial strains were grown in CDM and incubated at 32° C. for 36 h to determine their ability to decarboxylate HCAs. Results in the below table show that three strains were able to decarboxylate pCA, CA and FA (Enterococcus mundtii, Lactobacillus plantarum, and Pediococcus pentosaceus). Six strains were able to decarboxylate pCA and CA (Lactobacillus helveticus Lactobacillus pentosus, Lactobacillus plantarum, Pediococcus acidilactici Pediococcus pentosaceus). Two strains were able to decarboxylate only pCA (Lactobacillus acidophilus and Lactobacillus helveticus); and one strain was able to decarboxylase only CA (Lactobacillus helveticus). No strain examined showed the ability to decarboxylate SA under these conditions. However, this behavior was not unexpected, as literature shows that LAB may not be capable of SA decarboxylation. Moreover, this selectivity against SA is apparently not unique to bacterial PAD, as yeast-derived PAD seems to have a similar behavior (Edlin, D. A. N., Narbad, A.; Gasson, M. J.; Dickinson, J. R.; Lloyd, D. Purification and characterization of hydroxycinnamate decarboxylase from Brettanomyces anomalus. Enzym, Microb. Technol. 1998, 22, 232-239), Although Enterococcus mundtii, Pediococcus pentosaceus, and Lactobacillus plantarum were capable of decarboxylating all pCA, CA and FA; none was able to completely decarboxylate FA as they did with pCA and CA. These decarboxylation patterns for two of the three strains aforementioned are consistent with previous literature, where Lactobacillus plantarum and Pediococcus pentosaceus were able to metabolize pCA and CA with high efficiency, but FA only partially. However, no information on the decarboxylating capabilities of Enterococcus mundtii was found in published literature. This report showed the ability of the Enterococcus mundtii strain to decarboxylate HCAs, resulting in the production of 4VPs, strongly suggesting that this strain can in fact, synthesize PAD. This finding may promote the use of this strain for targeted bioconversion of hydroxycinnamic acids, with special interest in its capability to decarboxylate FA into 4-VG in products such as sourdough bread (Ripari, V.; Bai, Y.; Gänzle, M. G. Metabolism of phenolic acids in whole wheat and rye malt sourdoughs. Food Microbiol. 2019, 77, 43-51) and functional beverages obtained from lactic acid fermentation (Nsogning Dongmo, S.; Sacher, B.; Kollmannsberger, H.; Becker, T. Key volatile aroma compounds of lactic acid fermented malt based beverages—impact of lactic acid bacteria strains. hood Chem. 2017, 229, 565-573).

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

| Lactic Acid | | | | | |
|---|---|---|---|---|---|
| Bacteria Strain | Accession Code | pCA | CA | FA | SA |
| Enterococcus | OSU-PECh-39B | (++) | (++) | (+) | (−) |
| mundtii | | | | | |
| Lactobacillus | OSU-PECh-LA5 | (++) | (−) | (−) | (−) |
| acidophilus | | | | | |
| Lactobacillus | OSU-PECh-25 | (−) | (−) | (−) | (−) |
| helveticus | OSU-PECh-26 | (−) | (−) | (−) | (−) |
| | OSU-PECh-33 | (−) | (−) | (−) | (−) |
| | OSU-PECh-40 | (−) | (−) | (−) | (−) |
| | OSU-PECh-57B | (−) | (−) | (−) | (−) |
| | OSU-PECh-60 | (−) | (−) | (−) | (−) |
| | OSU-PECh-LH1B | (++) | (−) | (−) | (−) |
| | OSU-PECh-LH4A | (−) | (−) | (−) | (−) |
| | OSU-PECh-LH7 | (++) | (++) | (−) | (−) |
| | OSU-PECh-LH15A | (−) | (−) | (−) | (−) |
| | OSU-PECh-LH19 | (−) | (++) | (−) | (−) |
| Lactobacillus | OSU-PECh-LP6C | (++) | (++) | (−) | (−) |
| pentosus | | | | | |
| Lactobacillus | OSU-PECh-A | (++) | (++) | (−) | (−) |
| plantarum | OSU-PECh-BB | (++) | (++) | (+) | (−) |
| Lactobacillus | OSU-PECh-24 | (−) | (−) | (−) | (−) |
| rhamnosus | | | | | |
| Pediococcus | OSU-PECh-PA3A | (++) | (++) | (−) | (−) |
| acidilactici | OSU-PECh-PAL | (−) | (−) | (−) | (−) |
| Pediococcus | OSU-PECh-27B | (++) | (++) | (+) | (−) |
| pentosaceus | OSU-PECh-PP6A | (++) | (++) | (−) | (−) |
| | OSU-PECh-PP13 | (++) | (++) | (−) | (−) |
| Lactobacillus | OSU-PECh-C | (−) | (−) | (−) | (−) |
| casei | | | | | |
| Staphylococcus | ATCC 1222 | (−) | (−) | (−) | (−) |
| epidermidis | | | | | |

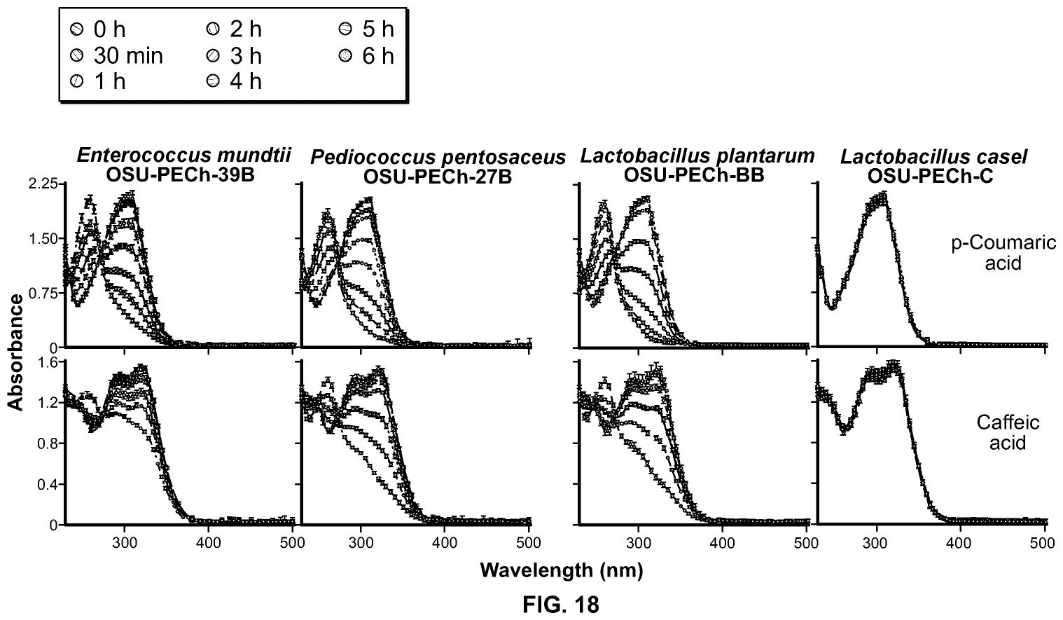

Moreover, in order to investigate if the light absorption changes were time dependent, samples were taken after 30 min of incubation and every hour thereafter, and their light absorption spectra were analyzed. FIG. 18 shows that the changes in the absorption spectra are in fact, time dependent and further analysis showed that pCA is decarboxylated at a faster rate than CA, with this trend being independent of the bacterial strain used. This is similar to a previous report using purified PAD, where at high concentrations of HCAs, pCA and CA were decarboxylated much faster than FA (Rodriguez, H.; Angulo, I.; De Rivas, B. L.; Campillo, N.; Páez, J. A.; Muñoz, Mancheño, J. M. p-Coumaric acid decarboxylase from Lactobacillus plantarum: Structural insights into the active site and decarboxylaction catalytic mechanism. Proteins 2010, 78, 1662-1676). A possible explanation for this behavior may be related to the antibacterial effect of each HCA. Reports show that against E. coli, S. aureus and B. cereus, and LAB, pCA had a stronger antibacterial activity than a more polar CA and a less polar FA. Moreover, it was hypothesized that this stronger antibacterial activity may be related to the solubility of pCA in both aqueous and lipid phases. This particular solubility pattern allows pCA to bind to the outer bacterial membrane, facilitating its interaction and subsequent decarboxylation by PAD.

### Materials and Methods

### Chemicals and Reagents

CA, FA, SA, dipotassium hydrogen phosphate (K₂HPO₄), monopotassium phosphate (KH₂PO₄), yeast extract, magnesium sulfate (MgSO₄) and glucose were purchased from Sigma-Aldrich (St. Louis, Mo., USA); pCA was purchased from MP Biomedicals (Solon, Ohio, USA); sodium chloride (NaCl) was obtained from Fisher Scientific (Pittsburgh, Pa., USA); MRS lactobacilli broth was purchased from Difco Laboratories (Detroit, Mich., USA). 4VP was purchased from Santa Cruz Biotechnology (Santa Cruz, Calif., USA). All other reagents and solvents were of analytical grade.

### Growth Medium Selection for HCA Decarboxylation Experiments

To develop a rapid, high-throughout spectrophotometric method, a medium with minimal light absorption interference at the wavelengths of interest had to be selected. The light absorption spectra of two growth media were compared; commercially available MRS broth, commonly used as a LAB growth medium; and CDM, designed to provide LAB with the minimum required nutrients for normal growth (Rocha-Mendoza, Kosmerl, E.; Miyagusuku-Cruzado, G.; Giusti, M. M.; Jiménez-Flores, R.; García-Cano, I. Growth of lactic acid bacteria in milk phospholipids enhances their adhesion to Caco-2 cells. J. Dairy Sci. 2020). CDM was prepared using 0.1% KH₂PO₄, 0.1% KH₂PO₄, 0.5% yeast extract, 0.025% MgSO₄, 0.0005% NaCl and 0.5% glucose in distilled water. Absorption spectra in the wavelengths of interest (200-500 nm) were obtained using a SpectraMax M2 plate reader (Molecular Devices, Sunnyvale, Calif., USA) after diluting the growth medium 25 times with 0.1 M phosphate buffer pH 7.0.

### Bacterial Strain Selection

LAB strains were isolated from dairy products such as milk, milk powder, buttermilk powder, cheese and yogurt (García-Cano, I.; Rocha-Mendoza, D.; Ortega-Anaya, J.; Wang, K.; Kosmerl, E.; Jiménez-Flores, R. Lactic acid bacteria isolated from dairy products as potential producers of lipolytic, proteolytic and antibacterial proteins. Appl. Microbiol. Biotechnol. 2019, 103, 5243-5257). Initial selection from the 137 LAB strains in the was conducted using bioinformatic tools from the National Center for Biotechnology Information (NCBI, https://www.ncbi.nlm.nih.gov/) and genome annotations for each species (Pruitt, K. D.; Tatusova, T.; Maglott, D. R. NCBI reference sequences (RefSeq): A curated non-redundant sequence database of genomes, transcripts and proteins. Nucleic Acids Res. 2007. 35, 61-65). Bacterial strains were selected based on their potential capability to synthesize phenolic acid decarboxylase (PAD), an enzyme responsible for metabolizing HCAs, which has been previously purified, characterized and sequenced (Cavin, J. F.; Dartois, V.; Diviès, C. Gene cloning, transcriptional analysis, purification, and characterization of phenolic acid decarboxylase from Bacillus subtilis. Appl. Env. Microbiol. 1998, 64, 1466-1471). Positive and negative controls for PAD synthesis were initially selected using the aforementioned methodology, and the PAD activity of these strains was verified using a previously published spectrophotometric method and pCA as a decarboxylation substrate. Based on these preliminary studies, Staphylococcus epidermidis was selected as a non-LAB strain, with no PAD activity (non-LAB negative control). Lactobacillus casei isolated from whey protein isolate produced by Hilmar Ingredients (Hilmar, Calif., USA), was selected as a LAB strain showing no PAD activity (LAB negative control). Lactobacillus plantarum isolated from natural yogurt produced by Superior Dairy (Canton, Ohio, USA) showing PAD activity and was selected as a LAB positive control.

### Decarboxylation Experiments

Preparation of stock solutions and incubation in CDM was conducted using a modification of a previously reported spectrophotometric method. Briefly, stock solutions of pCA, CA, FA and SA were prepared at a concentration of 5 g/L in aqueous ethanol (50% v/v). Stock solutions of HCAs were diluted in CDM for a final concentration of 500 mg/L. This allowed for a clear identification of absorption changes, as the spectrum is initially dominated by the HCA and after incubation, by the degradation product. Bacterial strains potentially capable of producing PAD were initially grown in MRS broth in sterile centrifuge tubes under microaerobic conditions, and incubated overnight at 32° C. The culture was collected by centrifugation at 8000× g for 10 min at 4° C. (Centrifuge 5804R, Eppendorf, Boulder, Colo., USA), the supernatant was then discarded, and pellets were washed twice with sterile saline solution (0.085% NaCl, pH 7.0). Washed pellets were reconstituted in sterile saline solution and seeded on 96-well plates with CDM containing HCAs. LAB strains were seeded at an optical density of 0.1 (O.D. 600 nm) calculated using a SpectraMax M2 plate reader (Molecular Devices, Sunnyvale, Calif., USA). Plates were covered with a sterile lid, sealed with parafilm to avoid evaporation, and were then incubated at 32° C. for 36 h. Each HCA used in this study was incubated individually with every LAB strain.

Rapid, High-Throughout Screening of Hydroxycinnamic acid Degradation by LAB Using UV-Vis Spectroscopy and Selection of Optimal pH for Measurement

Spectrophotometric analyses were used to monitor the ability of bacteria to decarboxylate HCAs. Briefly, 96-well plates were centrifuged at 4000× g for 30 min at room temperature (Eppendorf 5804 benchtop centrifuge, Eppendorf, Boulder, Colo., USA, Eppendorf A-2-DWP microplate rotor). Supernatants were then immediately collected and frozen until further analysis. To select the best pH for monitoring absorption changes, a 10 μL aliquot of the CDM containing HCAs or their decarboxylated products, was diluted 25 times with either KCl buffer (0.25 M) at pH 1.0, citrate buffer (0.1 M) at pH 3.0 or 5.0, or phosphate buffer (0.1 M) at pH 7.0. Subsequently, the diluted aliquots were placed in a UV-transparent 96-well microplate (Thermo Scientific Nunc®, Wilmington, Del., USA) for the measurement of their absorbance between the range of 230-500 nm with a SpectraMax M2 plate reader (Molecular Devices, Sunnyvale, Calif., USA) at 25° C.

uHPLC-DAD-ESI-MS Analysis of HCAs and Degradation Products

A Nexera-i-LC-2040 3D ultra-high-pressure liquid chromatograph was used for reverse-phase chromatographic separation and UV-Vis light absorption characterization. This system consisted of 4 pumps, a refrigerated autosampler, a column oven, and a diode-array detector (DAD). Tentative identification of HCAs and their degradation products was conducted using a LCMS-8040 triple quadrupole mass spectrometer with electrospray interface (Shimadzu, Columbia, Md., USA). Briefly, 30 μL of supernatant were injected into a Restek Pinnacle DB C18 column (50×2.1 mm, 1.9 μm, Restek, Bellefonte, Pa., USA) using a binary solvent system consisting of 0.1% aqueous formic (A) and acetonitrile (B). The elution profile was 0-10 min, 3-20% B in A, 10-11 min, 20-40% B in A, 11-13 min, 40% B (isocratic), with a flow rate of 0.3 mL/min at 40° C. Identical ionizing conditions for the electrospray interface were used for tentative identification of degradation products: 1.5 L/min nebulizing gas, 230° C. desolvation line temperature, 300° C. heat block temperature, 15 L/min drying gas flow. MH⁺ and M⁻ of intact structures were analyzed under positive and negative mode, respectively, using the Q1 scan function from 100 to 1000 m/z and single ion monitoring function for the respective 4VPs and 4-ethylphenols at their characteristic m/z.

### Statistical Analysis

All experiments were conducted in triplicate. Figure design, one-way ANOVA is analyses and Tukey post hoc tests were conducted using GraphPad Prism 8.3.0 (GraphPad Software, San Diego, Calif., USA). A value of p<0.05 was considered significant.

### Conclusion

The decarboxylating capabilities of a collection of LAB were successfully studied. with a fast, simple, and reproducible methodology based on UV-Vis light absorption changes. Results were consistent with the formation of 4VPs, decarboxylated products from pCA, CA and FA, while no decarboxylation was observed for SA. In this study, we reported the ability of the Enterococcus mundtii strain to decarboxylate pCA, CA and FA; strongly suggesting that this strain is capable of synthesizing PAD. This rapid and inexpensive method has an advantage of using reagents, materials, and equipment that can be found in most laboratories. Moreover, the selection of a growth medium with low interference and the correct pH for the detection of hypsochromic changes, allow for a clearer visualization of the results. This high-throughout method will facilitate the screening of LAB strains or fermentation starters capable of enzymatic decarboxylation of different HCAs, as it does not require further preparations or extraction procedures. Furthermore, LAB grown in CDM can potentially be used for the production of 4VPs as it does not seem to promote the activity of a phenyl reductase, evidenced by the absence of 4-ethylphenol after incubation. This method can help in the selection of bacterial strains that capable or incapable, depending on the application, of decarboxylating HCAs into 4VPs, for their use in industrial fermentation processes. Ultimately, the simple, rapid method and the information conveyed in this report can promote the selection of better fermentation starters capable of targeted HCA biotransformation into valuable flavor compounds. This could result in significant quality improvements in the flavor and color of fermented products and an increased consumer acceptance. Future experiments will focus on the effect of the matrix composition and physicochemical parameters on the decarboxylating capabilities of Lactobacillus plantarum, Pediococcus pentosaceus and Enterococcus mundtii in different food matrices. Moreover, future studies will also focus on the expression, purification, and characterization of the PAD from Enterococcus mundtii.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of ” and “consisting of ” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method for preparing one or more pyranoanthocyanins of Formula I 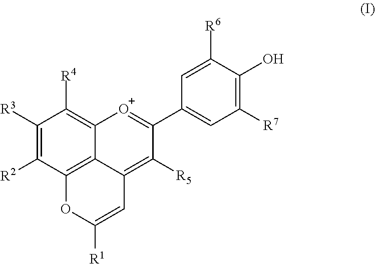
wherein:
R¹ is selected from the group consisting of: 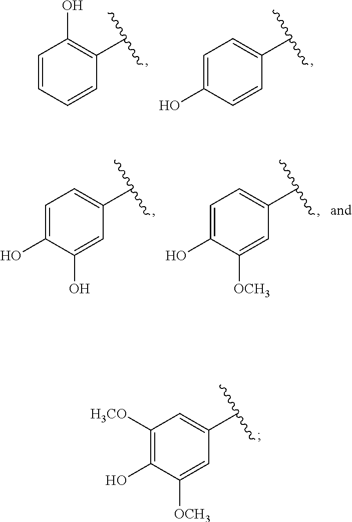 [SMILES: *c1ccccc1O ; *c1ccc(O)cc1 ; *c1ccc(O)c(O)c1 ; *c1ccc(O)c(OC)c1 ; *c1cc(OC)c(O)c(OC)c1]
R² is selected from —H and —OCH₃;
R³ is selected from —OH and —OCH₃;
R⁴ is selected from —H and —OCH₃;
R⁵ is selected from —H, —OH, —O-acyl, and a non-acylated or acylated O-glycoside;
R⁶ is selected from —H, —OH, and —OCH₃; and
R⁷ is selected from —H, —OH, and —OCH₃;
the method comprising:
reacting one or more compounds of Formula II 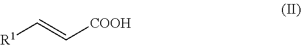
with a bacterium expressing a phenolic acid decarboxylase to form one or more compounds of Formula III 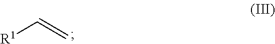
and
reacting the one or more compounds of Formula III with one or more compounds of Formula IV 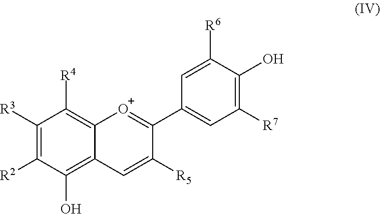
at a temperature ranging from about 35 degrees Celsius to about 45 degrees Celsius to form the one or more pyranoanthocyanins of Formula IV.

2-6. (canceled)

7. The method of claim 1, wherein R² is —H.

8. The method of any claim 1, wherein R³ is —OH.

9. The method of claim 1, wherein R⁴ is —H.

10-15. (canceled)

16. The method of claim 1, wherein R⁵ is —H.

17. The method of claim 1, wherein R⁵ is —OH.

18. The method of claim 1, wherein R⁵ is an —O-(acyl) group selected from —O-acetyl, —O-(malonyl), —O-(succinyl), —O-(4-hydroxybenzoyl), —O-(3,4-dihydroxybenzoyl), —O-(vanilloyl), —O-(galloyl), —O-(syringoyl), —O-(2-coumaryl), —O-(4-coumaryl), —O-(caffeoyl), —O-(feruloyl), and —O-(sinapoyl).

19. The method of claim 1, wherein R⁵ is a non-acylated O-glycoside selected from arabinoside, galactoside, glucoside, glucosyl(1→2)glucoside, rhamnosyl(1→6)glucoside, xylosyl(1→2)galactoside, xylosyl(1→2)glucoside, glucosyl(1→6)galactoside, and xylosyl(1→2)glucosyl(1→6)galactoside.

20. The method of claim 1, wherein R⁵ is an acylated O-glycoside selected from arabinoside, galactoside, glucoside, glucosyl(1→2)glucoside, rhamnosyl(1→6)glucoside, xylosyl(1→2)galactoside, xylosyl(1→2)glucoside, glucosyl(1→6)galactoside, and xylosyl(1→2)glucosyl(1→6)galactoside substituted with one or more acyl groups independently selected from acetyl, malonyl, succinyl, 4-hydroxybenzoyl, 3,4-dihydroxybenzoyl, vanilloyl, galloyl, syringoyl, 2-coumaryl, 4-coumaryl, caffeoyl, feruloyl, and sinapoyl.

21. The method of claim 1, wherein the bacterium is a lactic acid bacterium selected from a Lactobacillus species, a Leuconostoc species, a Pediococcus species, a Lactococcus species, a Streptococcus species, and an Enterococcus species.

22. (canceled)

23. A method for preparing one or more pyranoanthocyanins of Formula I: 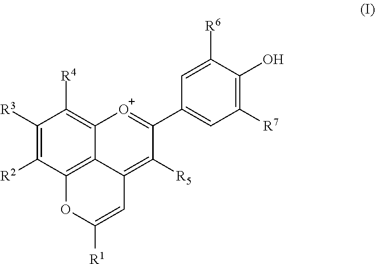
wherein:
R¹ is selected from the group consisting of: 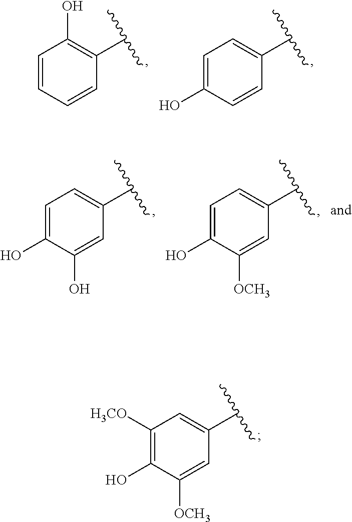 [SMILES: *c1ccccc1O ; *c1ccc(O)cc1 ; *c1ccc(O)c(O)c1 ; *c1ccc(O)c(OC)c1 ; *c1cc(OC)c(O)c(OC)c1]
R² is selected from —H and —OCH₃;
R³ is selected from —OH and —OCH₃;
R⁴ is selected from —H and —OCH₃;
R⁵ is selected from —H, —OH, —O-acyl, and a non-acylated or acylated O-glycoside;
R⁶ is selected from —H, —OH, and —OCH₃; and
R⁷ is selected from —H, —OH, and —OCH₃;
the method comprising:
reacting one or more compounds of Formula II 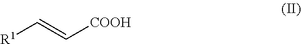
with one or more compounds of Formula IV 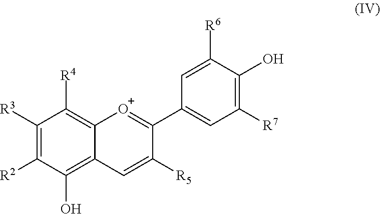
at a temperature ranging from about 30 degrees Celsius to about 80 degrees Celsius to form the one or more pyranoanthocyanins of Formula I.

24-28. (canceled)

29. The method of claim 23, wherein R² is —H.

30. The method of claim 23, wherein R³ is —OH.

31. The method of claim 23, wherein R⁴ is —H.

32-37. (canceled)

38. The method of claim 23, wherein R⁵ is —H.

39. The method of claim 23, wherein R⁵ is —OH.

40. The method of claim 23, wherein R⁵ is an —O-(acyl) group selected from —O-acetyl, —O-(malonyl), —O-(succinyl), —O-(4-hydroxybenzoyl), —O-(3,4-dihydroxybenzoyl), —O-(vanilloyl), —O-(galloyl), —O-(syringoyl), —O-(2-coumaryl), —O-(4-coumaryl), —O-(caffeoyl), —O-(feruloyl), and —O-(sinapoyl).

41. The method of claim 23, wherein R⁵ is a non-acylated O-glycoside selected from arabinoside, galactoside, glucoside, glucosyl(1→2)glucoside, rhamnosyl(1→6)glucoside, xylosyl(1→2)galactoside, xylosyl(1→2)glucoside, glucosyl(1→6)galactoside, and xylosyl(1→2)glucosyl(1→6)galactoside.

42. The method of claim 23, wherein R⁵ is an acylated O-glycoside selected from arabinoside, galactoside, glucoside, glucosyl(1→2)glucoside, rhamnosyl(1→6)glucoside, xylosyl(1→2)galactoside, xylosyl(1→2)glucoside, glucosyl(1→6)galactoside, and xylosyl(1→2)glucosyl(1→6)galactoside substituted with one or more acyl groups independently selected from acetyl, malonyl, succinyl, 4-hydroxybenzoyl, 3,4-dihydroxybenzoyl, vanilloyl, galloyl, syringoyl, 2-coumaryl, 4-coumaryl, caffeoyl, feruloyl, and sinapoyl.

43. The method of claim 23, wherein the compound of Formula IV is reacted with the compound of Formula II at a molar ratio of about 1:50 to about 1:200 (Formula IV:Formula II).
